(19) United States
(12) Patent Application Publication
(10) Pub. No.: US 2022/0008918 A1
(43) Pub. Date: Jan. 13, 2022

(54) METHODS AND DEVICES FOR PERFORMING FLOW-THROUGH CAPTURE OF LOW-CONCENTRATION ANALYTES

(71) Applicant: CALIFORNIA INSTITUTE OF TECHNOLOGY, Pasadena, CA (US)

(72) Inventors: Rustem F. ISMAGILOV, Altadena, CA (US); Travis S. SCHLAPPI, Pasadena, CA (US); Stephanie E. MCCALLA, Bozeman, MT (US); Nathan G. SCHOEPP, Pasadena, CA (US)

(21) Appl. No.: 16/316,193

(22) PCT Filed: Jul. 7, 2017
(86) PCT No.: PCT/US2017/041172

§ 371 Date: Jan. 8, 2019

Related U.S. Application Data
(60) Provisional application No. 62/360,272, filed on Jul. 8, 2016.

(51) Int. Cl. B01L 3/00; C12Q 1/686; B01L 7/00

(52) U.S. Cl. CPC B01L 3/502715; B01L 3/5023; B01L 2300/069; B01L 7/52; B01L 2300/163; C12Q 1/686

(57) ABSTRACT

Methods and devices for detecting a low concentration analyte in a sample are provided herein. The methods include flowing a sample through a porous membrane coated with a capture matrix to capture the low concentration analyte. The methods also can include detecting the captured analyte, such as by performing in-situ amplification of the analyte.

### CROSS-REFERENCE TO RELATED APPLICATIONS

This application is the National Stage of International Application No. PCT/US2017/041172, filed Jul. 7, 2017, which claims the benefit of and priority to U.S. Provisional Patent Application No. 62/360,272, filed Jul. 8, 2016, the contents of which are incorporated herein by reference in their entirety.

### STATEMENT REGARDING FEDERALLY SPONSORED RESEARCH OR DEVELOPMENT

This invention was made with government support under Grant No. HR0011-11-2-0006 awarded by DARPA. The government has certain rights in the invention.

### INTRODUCTION

Capture of analytes from solution, such as by filtration, is a commonly applied biological assay technique. Such capture can include concentrating analytes by passing the analytes in solution over or through a capture matrix coating a support structure, e.g. a membrane. The capture matrix in turn restricts the movement of the analytes away from the coated membrane without restricting the movement of the remaining solution. One practical application of analyte capture is the concentration of nucleic acids by filtration into volumes, e.g., a few μLs, that are amenable to subsequent manipulation, e.g., amplification processes, such as PCR and/or LAMP. In such a circumstance, analytes having even very small, e.g., zeptomolar, initial concentrations in solution can be captured from a solution and thereby concentrated.

### SUMMARY

Methods and devices for detecting a low concentration analyte in a sample are provided herein. The methods include flowing a sample through a porous membrane coated with a capture matrix to capture the low concentration analyte. The methods also can include detecting the captured analyte, such as by performing in-situ amplification of the analyte.

In various aspects, the methods are methods of detecting a low concentration analyte in a sample and include flowing a sample including the low concentration analyte through a porous membrane coated with a capture matrix and thereby capturing analyte with the membrane. The methods can also include detecting the captured analyte. In some aspects, the analyte has a concentration within the sample of 500 entities/mL or less, or 100 entities/mL or less or of 10 entities/mL or less. Also, in some aspects, the flowing is performed in 1 hour or less or 30 min or less or 10 min or less.

In some versions, the sample is flowed through the coated membrane at a rate of 0.1 mL/minute or greater, 0.5 mL/minute or greater or 1 mL/minute or greater. Also, in various aspects, the sample has a volume of 0.1 mL or greater, 1 mL or greater, or 20 mL or greater.

According to some embodiments, the analyte includes nucleic acids, bacteria, viruses, and/or cells. Also, in some aspects, detecting the captured analyte includes performing nucleic acid amplification. In various aspects, the coated membrane is in a container and the nucleic acid amplification is performed while the captured analyte is in the container.

In some embodiments, the membrane is coated with a matrix composed of a polymeric material such as poly-L-lysine, and/or chitosan. In various aspects of the methods, flowing the sample through the coated membrane includes concentrating the sample on the membrane by 1000× or more.

According to various aspects, the methods are methods of performing in-situ amplification on a sample. Such methods can include flowing the sample having a first concentration of an analyte through a porous membrane coated with a capture matrix in a container and thereby capturing analyte with the membrane to provide a captured sample having a second concentration of analyte which is 1000× or more than the first concentration. Such methods can also include amplifying the analyte within the container, wherein the flowing and amplifying are performed in 1 hour or less. In such methods, the first concentration can be 100 entities/mL or less, or 10 entities/mL or less. Also, according to some aspects, the methods include performing flowing and/or amplifying in 30 min or less, such as in 10 min or less.

In various aspects, the sample is flowed through the coated membrane at a rate of 0.1 mL/minute or greater or 0.5 mL/minute or greater, or 1 mL/minute or greater. Also, in some versions, the sample has a volume of 0.1 mL or greater, or 1 mL or greater, or 20 mL or greater.

According to embodiments of the methods, amplifying the analyte includes performing nucleic acid amplification. Also, in some aspects, the membrane is coated with a matrix including chitosan and/or a polymeric material such as poly-L-lysine.

In some versions, the methods are methods of performing flow-through capture of nucleic acids with a porous membrane coated with a capture matrix. Such aspects can include flowing a nucleic acid amplification sample including nucleic acids and having a first concentration through a porous membrane coated with a capture matrix and thereby capturing one or more of the nucleic acids with the matrix to provide a captured sample. In some aspects, the captured sample has a second concentration which is 1000× or more than the first concentration, and/or in some aspects the flowing is performed in 30 min or less, such as in 10 min or less.

According to various aspects, the methods include detecting nucleic acids in the captured sample by performing nucleic acid amplification. Also, in some aspects, the porous membrane coated with a capture matrix is in a container and the nucleic acid amplification is performed without removing the captured nucleic acids from the container. A membrane can be coated with a matrix including chitosan and/or a polymeric material, e.g., poly-L-lysine.

In some embodiments, a membrane is cylindrical and has a membrane radius of 2 mm or less. Also, in various aspects, the membrane has a pore radius ranging from 0.5 to 20 μm and/or a thickness ranging from 0.3 to 3500 μm. According to some aspects of the methods, the sample is flowed through the coated membrane at a rate of 0.1 mL/minute or greater, such as 0.5 mL/minute or greater, such as 1 mL/minute or greater.

Furthermore, the subject embodiments also include devices including low concentration analyte capture devices. In various aspects, the devices include a housing and/or a coated membrane operatively coupled to the housing and configured to capture and thereby concentrate analyte from a sample flowed therethrough by 1000× or more in a time period, such as in 30 min or less. In various aspects, the housing includes a container and the coated membrane is positioned within the container.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIGS. 1A-1C provide a theoretical model and numerical simulations for flow-through capture. More specifically, FIG. 1A provides a schematic drawing showing the process of capturing nucleic acids from a sample flowing through a porous membrane (which has been coated with a capture matrix). FIG. 1B provides predictions for the percentage of molecules captured at the pore wall as a function of the Damkohler number (Da). FIG. 1C provides predictions for the percentage of molecules captured at the pore wall as a function of the Peclet number (Pe). Pe is changed by varying the velocity (U), pore length (δm), or pore diameter (Rp); all result in a similar dependence of capture percentage on Pe.

FIG. 2 provides a schematic diagram of flow-through simulation geometry. The shaded portions labelled “A and B” represent the capture matrix (γ) coated on the surface, e.g., interior surface, of the pore wall.

FIGS. 3A and 3B provides graph of membrane radius, pore radius, and membrane thickness tradeoffs for achieving high flow rates while also maintaining reasonable pressure drop (ΔP) and a low Peclet number (Pe). More specifically, FIG. 3A provides combinations of membrane radius, pore radius, and flow rate that maintain Pe<1 for different membrane thicknesses. Any point below the surface curvature has Pe<1. FIG. 3B provides the influence of membrane and pore radius on pressure drop with the flow rate through the membrane held constant at 1 mL/min. The overlap of the triangle at the upper left of each plot (Pe<1) and the darkened area represents efficient and rapid capture with a reasonable pressure drop (ΔP<1 atm). The white area signifies a combination of membrane and pore radius that results in prohibitively large pressure drops (ΔP>1 atm) necessary to achieve 1 mL/min.

FIG. 4 provides a diagram illustrating how capture efficiency depends on flow rate.

FIG. 5 provides a graph of DNA binding capacity of chitosan-coated membranes.

FIGS. 6A and 6B provide graphs of compatibility of chitosan membranes with PCR and LAMP amplification. More specifically, FIG. 6A provides dilutions of λ DNA which were wetted onto chitosan membranes or placed into a well plate without a membrane; PCR mix was added and amplification was detected via melt curve analysis. Six replicates were run at each dilution; the percent of replicates positive for λ DNA product is shown (n=6). FIG. 6B provides 20 copies of λ DNA were wetted onto chitosan membranes within a well plate, or placed into a well plate without a membrane; LAMP mix was added and amplification was detected via real-time fluorescence. Three replicates were run for each sample; the fluorescent traces as a function of time are plotted.

FIGS. 7A and 7B provide schematic diagrams of capture and in situ amplification. More specifically, FIG. 7A provides nucleic acids in a solution with pH<6.3 will electrostatically bind to the protonated chitosan-coated pore wall. FIG. 7B provides addition of amplification mix (pH˜8) deprotonates the chitosan and releases nucleic acids. Thermal cycling amplifies DNA.

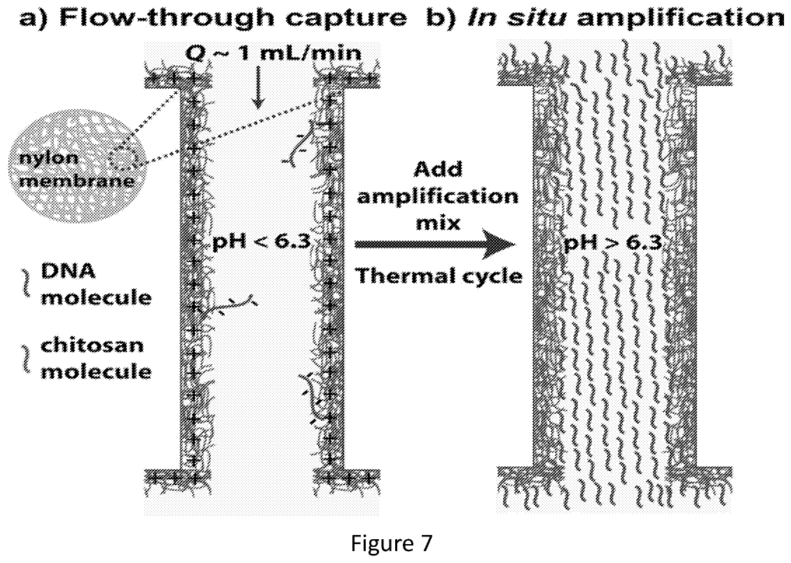

FIGS. 8A-8E provide a schematic of a syringe/luer lock system used to flow mL-scale volumes through chitosan membranes with a diameter of 4 mm. A chitosan membrane is placed in between two luer locks. A syringe containing a nucleic acid sample is connected to the top luer lock and the plunger is compressed to flush the sample through the membrane. Then, the luer locks are disconnected from the syringe, taken apart, and the membrane containing captured nucleic acids is placed in a PCR tube along with amplification mix for thermal cycling.

FIGS. 9A and 9B provide graphs illustrating nucleic acid detection. More specifically, FIG. 9A provides a graph illustrating nucleic acid detection via flow-through capture and in situ amplification on chitosan membranes. Percent of membranes that were positive for λ DNA product over different experiments on different days at a concentration of 0.5 copies/mL target DNA (25 copies of λ DNA in 50 mL of 10 mM MES buffer) and 10 or 100 ng background DNA added. The volume flowed through was 50 mL (Table S-5). Each bar of the graph represents a percentage positive of 9 samples (for 10 ng background DNA) or 10 samples (for 100 ng background DNA). Error bars are 1 S.D. Table S-5 shows all the quantities and concentrations of λ DNA, volumes of 10 mM MES buffer, and amounts of background DNA added to generate FIG. 9A. FIG. 9B provides a graph illustrating nucleic acid detection via flow-through capture and in situ amplification on chitosan membranes. Percent of membranes that were positive for λ DNA product over different experiments on different days for varying concentrations (0.9-6.0 copies/mL). The volume flowed through ranged from 1 to 10 mL (Table S-4). Each bin of the histogram has 9-15 samples for a total of 24 samples. Error bars are 1 S.D. Table S-4 shows all the quantities of λ DNA, volumes of 10 mM MES buffer, and concentrations used to generate FIG. 9B. Table S-4 shows all the quantities of λ DNA, volumes of 10 mM MES buffer, and concentrations used to generate FIG. 9B.

FIGS. 10A and 10B provide illustrations of DNA detection after in situ amplification. More specifically, FIG. 10A provides varying concentrations of λ DNA in 10 mM MES buffer were flowed through chitosan membranes. The membranes were then placed in a well plate and thermal cycled. After thermal cycling, each sample was run on a gel. Lanes 1-2: 5 copies/mL; Lanes 3-4: 2.5 copies/mL; Lane 5: positive control (10 copies of λ DNA in PCR mix, no membrane); Lane 6: negative control (0 copies of λ DNA in PCR mix, no membrane). FIG. 10B provides dilutions of λ DNA were wetted onto chitosan membranes; PCR mix was added and melt curve fluorescent traces are plotted. Three replicates were run at each dilution.

FIG. 11 provides a graph illustrating nucleic acid detection from human blood plasma via flow-through capture and in situ amplification on chitosan membranes. Percent of membranes that were positive for λ DNA product over different experiments on different days for varying concentrations (2-270 copies/mL) is provided. The volume flowed through ranged from 2 to 20 mL (Table S-6). Each bin of the histogram has 7-17 samples for a total of 38 samples. Error bars are 1 S.D. Table S-6 shows all the quantities of λ DNA, volumes of human blood plasma, and final concentrations of λ DNA used to generate FIG. 11.

### DETAILED DESCRIPTION

Methods and devices for detecting a low concentration analyte in a sample are provided herein. The methods include flowing a sample through a porous membrane coated with a capture matrix to capture the low concentration analyte. The methods also can include detecting the captured analyte, such as by performing in-situ amplification of the analyte.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Certain ranges can be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided can be different from the actual publication dates which can need to be independently confirmed.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

### Methods

Methods of detecting a low concentration analyte, e.g., an ultra-low concentration analyte, in a sample are provided herein. For example, in various embodiments, an analyte has a concentration within a sample of 500 entities/mL or less. Such methods, in various aspects, include flowing a sample including the low concentration analyte through a porous membrane coated with a capture matrix and thereby capturing analyte with the capture matrix. The flowing can be performed at a high flow rate such that the flowing is performed in 1 hour or less, such as 10 min or less. In various embodiments, the methods also include detecting the captured analyte, such as by amplifying and then detecting the captured analyte.

Analytes, according to the subject disclosure, can include nucleic acids such as free DNA and/or RNA, or any forms thereof, cells or cell portions, viruses, (e.g., HIV and/or HCV), bacteria, fungi, prions, and/or spores, or any combination thereof. Analytes can be single molecules, e.g., ketones, sugars such as glucose and/or polymers, or can be composites composed of a plurality of molecules, e.g., duplex DNA, protein complexes, viruses, cells or cell portions.

According to the subject disclosure, analytes can include RNA. In some cases, the RNA includes mRNA. In some cases, the RNA includes noncoding RNA (ncRNA). The noncoding RNA can include transfer RNA (tRNA), ribosomal RNA (rRNA), transfer-messenger RNA (tmRNA), small nucleolar RNA (snoRNA), microRNA (miRNA), small interfering RNA (siRNA), small nuclear RNA (snRNA), piwi-interacting RNA (piRNA), long ncRNA (IncRNA), and/or other types of ncRNA. In some versions, the RNA is from bacteria or viruses. In some versions, the RNA is collected from a cell.

Also, according to the embodiments a DNA analyte may be ssDNA, dsDNA, cDNA, or any combination thereof. In some aspects, the DNA includes a gene or a gene fragment. The gene or gene fragment can include a mutation. In some aspects, the DNA includes a non-coding region. In some aspects, the DNA includes cDNA. In some aspects, the DNA is from bacteria or viruses. In some aspects, the DNA is collected from a cell.

In some aspects, analytes can include proteins, fragments of proteins, or aggregates of proteins. The proteins can include TNF-alpha. The proteins can include glial fibrillary acidic protein (GFAP). The protein can include p24. In some cases, the proteins include enzymes. In some cases, the proteins include signaling proteins. In some cases, the proteins include membrane proteins. The membrane proteins can include receptor proteins, transport proteins, membrane enzymes, cell adhesion proteins, lipoproteins, and/or other membrane proteins. In some cases, the proteins include antibodies. The antibodies can include extracellular or membrane-associated proteins. In some cases, the proteins include ligand transport proteins. The ligand transport proteins can include hemoglobin, a carbohydrate binding protein, other ligand transport proteins, or any combination thereof. Examples of carbohydrate-binding proteins include, but are not limited to, lectins (such as, mannose-binding lectin (MBL)), collectins, pentraxin family members, ficolin, maltose-binding protein, arabinose-binding protein, and glucose-binding protein. The ligand transport proteins can include transmembrane proteins, such as ion channels. In some cases, the proteins include structural proteins. The structural proteins can include fibrous proteins, including collagen, elastin, keratin, or any combination thereof. The structural proteins can include globular proteins, including actin and tubulin monomers. The structural proteins can include motor proteins, including myosin, kinesin, dynein, or any combination thereof. In some cases, the protein is from a bacterium or from a virus. In some cases, the protein is collected from a cell. In some cases, the proteins include lipoproteins, including but not limited to high density lipoproteins and low density lipoproteins. In some cases, the proteins include tau or phosphorylated tau proteins.

In various embodiments, analytes can include peptides. In some cases, the peptides include tachykinin peptides. The tachykinin peptides can include substance P, kassinin, neurokinin A, neurokinin B, eledoisin, other tachykinin peptides, or any combination thereof. In some cases, the peptides include vasoactive intestinal peptides. The vasoactive intestinal peptides can include vasoactive intestinal peptide (VIP), pituitary adenylate cyclase activating peptide (PACAP), peptide histidine isoleucine 27 (PHI 27), growth hormone releasing hormone 1-24 (GHRH 1-24), glucagon, secretin, other vasoactive intestinal peptides, or any combination thereof. In some cases, the peptides include pancreatic polypeptide-related peptides. The pancreatic polypeptide-related peptides can include neuropeptide Y (NPY), peptide YY (PYY), avian pancreatic polypeptide (ΔPP), pancreatic polypeptide (PPY), other pancreatic polypeptide-related peptides, or any combination thereof. In some cases, the peptides include opioid peptides. The opioid peptides can include proopiomelanocortin (POMC) peptides, enkephalin pentapeptides, prodynorphin peptides, other opioid peptides, or any combination thereof. In some cases, the peptides include calcitonin peptides. The calcitonin peptides can include calcitonin, amylin, AGG01, other calcitonin peptides, or any combination thereof. In some cases, the peptides include other peptides. The other peptides can include B-type natriuretic peptide (BNP), lactotripeptides, other peptides, or any combination thereof. In some cases, the peptides can include Abeta. In some cases, the peptides are from a bacterium or from a virus. In some cases, the peptides are collected from a cell. Sometimes, the peptides are collected from the cell membrane. Occasionally, peptides are intracellular. In some cases, the peptides are extracellular. A peptide can be a protein.

In embodiments of the disclosure, analytes can include vesicles, including but not limited to exosomes, exosome-like vesicles, micro vesicles, epididimosomes, argosomes, microparticles, promininosomes, prostasomes, dexosomes, texosomes, dex, tex, archeosomes, and oncosomes. Analytes can include platelets. Analytes can include coagulation factors, including but not limited to Factor I, Factor II, Factor III, Factor IV, Factor V, Factor VI, Factor VII, Factor VIII, Factor IX, Factor X, Factor XI, Factor XII, Factor XIII, von Willebrand factor, prekallikrein (Fletcher factor), high-molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, cancer procoagulant, or combinations thereof.

In some aspects, analytes can include cells or fragments of cells. In some cases, the cells are bacterial. The bacterial cells can be collected from a culture, from a patient, from a surface, from the environment, from a biofilm, or from another source. The cells can include spores. The cells can include endospores. The cells can include anthrax spores. In some cases, the cells are prokaryotic. In some cases, the cells are eukaryotic. In some cases, the eukaryotic cells are human cells, or animal cells. The eukaryotic cells can be mammalian. A mammal can include, but is not limited to, a primate, ape, equine, bovine, porcine, canine, feline or rodent. A rodent can include, but is not limited to, a mouse, rat, or hamster.

In some versions, analytes can include viruses or viral particles (virions). Viruses can include, but are not limited to, norovirus, HIV, hepatitis C (HCV), common cold, influenza, chicken pox, ebola, and SARS. Analytes can include viral fragments. Analytes can include prions.

According to some versions, analytes can include metabolites. Analytes can include small molecules. Analytes can include carbohydrates. Analytes can include glycopatterns. Analytes can include specific glycopatterns on proteins. Analytes can include specific glycopatterns on cells.

A biological sample can be collected from a subject. Biological samples can include one or more cells. A biological sample can also not include one or more cells. In some embodiments, a biological sample can include free DNA, free RNA, viral particles, bacteria cells or cell portions, fungi, prions, spores, or any combination thereof. In certain embodiments, a subject is a “mammal” or a “mammalian” subject, where these terms are used broadly to describe organisms that are within the class mammalia, including the orders carnivore (e.g., dogs and cats), rodentia (e.g., mice, guinea pigs, and rats), and primates (e.g., humans, chimpanzees, and monkeys). In some embodiments, the subject is a human.

Furthermore, embodiments of the subject disclosure include sample preparation devices and methods of using the same, wherein the sample preparation devices including capture matrices supported on physical structures, e.g., membranes. As used herein, a “biological assay” is test on a biological sample that is performed to evaluate one or more characteristics of the sample or a portion thereof, e.g., an analyte. In certain implementations, the sample is a biological sample.

Accordingly, assay sample preparation devices, according to some embodiments, are devices that prepare a sample, e.g. a biological sample, for analysis with an assay. Also, in some aspects a biological sample is a nucleic acid amplification sample, which is a sample including one or more nucleic acids or portions thereof that can be amplified according to the subject embodiments.

In various embodiments, samples are prepared biological samples, e.g., biological samples which are prepared for processing, such as by amplification and/or further dowstream assaying. As such, in various aspects, the methods include preparing biological sample to, for example, produce a prepared biological assay sample. Aspects of the methods can include exposing a biological sample to a preparation solution, e.g., a cell lysing agent and/or a buffer, to produce a prepared biological assay sample. Producing the prepared biological sample can include exposing, such as by mixing in a container, a preparation solution to one or more aspects of the biological sample, wherein such exposure results in a change in the biological sample, e.g., cell lysing, such that the modified biological sample or a portion thereof, e.g., nucleic acids, can be further processed and/or analyzed, such as amplified.

In some embodiments of the subject disclosure, a prepared biological sample is a biological sample that has been processed by exposing the sample to a preparation solution, as described above. Such exposure can prepare the sample for binding to the capture matrix and can include lysing cells of the sample with a lysing agent of the preparation solution and/or extracting nucleic acids therefrom. Such extracted nucleic acids can be released into a resulting prepared sample solution. In some versions, the preparation solution is a nucleic acid amplification preparation solution and exposure to the solution prepares nucleic acids of the sample for amplification. After such exposure, the sample is a prepared nucleic acid amplification sample. In other embodiments, a prepared biological sample can include biological fluids, e.g. blood or urine, that have been subjected to centrifugation or size filtration.

As noted above, the methods include detecting a low concentration analyte, e.g., an ultra-low concentration analyte, in a sample. A low concentration analyte can have a concentration within the sample of, for example, 500 entities (e.g., molecules)/mL or less, 50 entities/mL or less, or 10 entities/mL or less or ranging from 0.01 entities/mL to 1000 entities/mL, inclusive. As used herein, “inclusive” refers to a provided range including each of the listed numbers. Unless noted otherwise herein, all provided ranges are inclusive. As used throughout this disclosure, where appropriate, the unit of entity/mL can be interchanged with molecules/mL, given that the analyte can be a single molecule, e.g., a nucleic acid such as DNA and/or RNA, or another entity, such as a virus, bacteria, cell, etc. As such, all of the numerical values listed with units of entity/mL also apply to molecules/mL, cells/mL, virions/mL and the like.

Furthermore, an analyte, e.g., a low concentration analyte, can have a concentration, e.g., a first and/or second concentration as described herein, within the sample of, for example, 1000 entities/mL or less, 500 entities (e.g., molecules)/mL or less, 400 entities/mL or less, 300 entities/mL or less, 250 entities/mL or less, 200 entities/mL or less, 100 entities/mL or less, 50 entities/mL or less, 40 entities/mL or less, 30 entities/mL or less, 25 entities/mL or less, 20 entities/mL or less, 10 entities/mL or less, 5 entities/mL or less, 1 entities/mL or less, 0.5 entities/mL or less, 0.4 entities/mL or less, 0.3 entities/mL or less, 0.2 entities/mL or less, 0.1 entities/mL or less, 0.05 entities/mL or less, or 0.01 entities/mL or less.

An analyte can also have a concentration, e.g., a first and/or second concentration as described herein, within the sample, for example, ranging from 0.01 entities/mL to 1000 entities/mL, such as from 0.05 entities/mL to 500 entities/mL, 0.1 entities/mL to 250 entities/mL, 0.1 entities/mL to 100 entities/mL, 0.1 entities/mL to 50 entities/mL, 0.1 entities/mL to 20 entities/mL, 0.1 entities/mL to 10 entities/mL, 0.1 entities/mL to 5 entities/mL, 0.1 entities/mL to 1 entities/mL, 0.2 entities/mL to 1 entities/mL, 0.2 entities/mL to 0.8 entities/mL, or 0.2 entities/mL to 0.5 entities/mL.

In various aspects, the second concentration is the concentration of analyte on, such as attached to, such as coupled and/or bonded to, such as ionically and/or covalently bonded to, and/or within a capture matrix, and within a volume defined by the surfaces of the membrane or other support structure. The second concentration may also be a concentration of analyte within a volume the same as, 100× or less, 500× or less, or 1000× or less that the volume of an initial sample, such as a sample having a first concentration, as described herein. The second concentration may also be a concentration of analyte within a volume defined by the surfaces of the coated structure and/or a volume that is within a distance of 1000 μm or less, 100 μm or less, 10 μm or less, 1 μm or less or 0.1 μm or less of the surfaces of the coated structure.

In some versions, the methods include capturing 100 or fewer, 75 or fewer, 50 or fewer, 40 or fewer, 30 or fewer, 25 or fewer, 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer analyte instances from a sample volume of 1 mL or less, or 5 mL or less, or 10 mL or less, or 25 mL or less, or 50 mL or less, or 1 mL or more, or 5 mL or more, or 10 mL or more, or 25 mL or more, or 50 mL or more.

Such methods, in various aspects, include flowing a sample including the low concentration analyte through a collecting element, e.g., a porous membrane coated with a capture matrix, and thereby capturing analyte with the coated membrane. Flowing the sample through the membrane can include flowing all, or substantially all, of the sample through the membrane. As used herein, “substantially” means to a great or significant extent, such as almost fully or almost entirely. Flowing the sample through the membrane can include moving a sample, e.g., a liquid sample, and/or e.g., a sample including a liquid substance such as water, and the analyte, through a first surface, e.g., a planar surface, of the membrane into the membrane. Flowing the sample through the membrane can also include moving sample or a portion thereof, e.g., a portion including a liquid substance such as water, without or substantially without the analyte, out of the membrane through a second surface e.g., a planar surface parallel to the first surface, of the membrane opposite the first membrane.

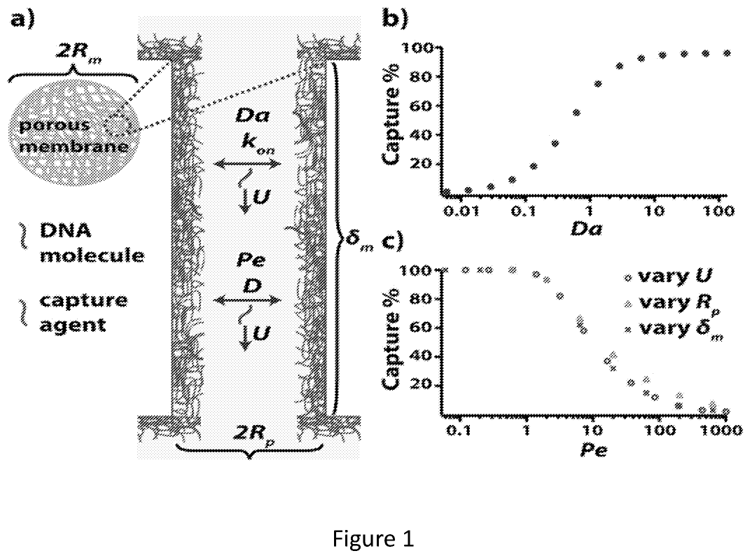

According to one operation of device use according to the subject methods an initial sample including a low, e.g., first, concentration analyte is flowed into the inlet and through the housing to contact and pass through a first surface of the porous membrane coated with a capture matrix. The coated membrane includes pores in it, each of which can be a pore as shown schematically in FIG. 1A. Analytes within the sample are retained within the coated membrane as the sample passes through the membrane. The remaining sample is then flowed out of the coated membrane through a second surface 1109 opposite the first surface 1108 and then through an outlet of the housing. The analyte retained by the coated membrane can then be amplified and/or detected while still within the housing.

Flowing the sample through the membrane can include capturing, such as by physically retaining and/or covalently and/or ionically bonding or otherwise retaining such as by trapping, analyte in the sample onto or into a capture matrix, while the depleted portion of the sample flows out of and away from the coated membrane. Such capturing can include retaining a high percentage, e.g., 70% or more, 80% or more, 90% or more, 95% or more, 97% or more, 98% or more, 99% or more, or 99.5% or more, of analyte with the coated membrane.

In various embodiments, a sample, e.g., a sample entering a membrane, has a first concentration of analyte. The methods can include flowing a sample having the first concentration into and through the membrane and thereby capturing analyte with the membrane to provide a captured sample, e.g., a sample having analyte on and/or in the membrane. Typically the volume of the sample far exceeds the interior volume of the membrane and therefore, a second concentration of analyte within the coated membrane is higher than the first concentration. In various aspects, the second concentration is 100× or more, such as 500× or more, such as 600× or more, such as 700× or more, such as 800× or more, such as 900× or more, such as 1000× or more, such as 1200× or more, such as 1500× or more, such as 1700× or more, such as 2000× or more, such as 2500× or more, such as 5000× or more than the first concentration. In some aspects, the second concentration ranges from 100× to 5000×, such as 500× to 2000×, 800× to 1500×, greater than the first concentration.

Furthermore, according to the subject disclosure, the methods can include flowing a sample through a device comprising a coated membrane, as described herein, at a rate of 0.01 mL/minute or greater, such as 0.05 mL/minute or greater, such as 0.1 mL/minute or greater, such as 0.5 mL/minute or greater, such as 1 mL/minute or greater, such as 2 mL/minute or greater, such as 5 mL/minute or greater. The methods can also include flowing a sample through a collecting element at a rate ranging from 0.05 mL/minute to 5 mL/minute, such as from 0.05 mL/minute to 1 mL/minute, such as from 0.05 mL/minute to 0.5 mL/minute, such as from 0.1 mL/minute to 5 mL/minute, such as from 0.1 mL/minute to 1 mL/minute, such as from 0.1 mL/minute to 0.5 mL/minute, or from 0.5 mL/minute to 2 mL/minute.

Also, in various embodiments, the sample, e.g., an initial sample or a captured sample, can have a volume of 0.02 mL or more, such as 0.05 mL or more, 0.1 mL or more, 0.5 mL or more, 1 mL or more, 3 mL or more, 5 mL or more, 10 mL or more, 20 mL or more, 25 mL or more, 30 mL or more, 40 mL or more, 50 mL or more, 75 mL or more, 100 mL or more, or 150 mL or more. In some embodiments, the sample, e.g., an initial sample or a captured sample, can have a volume of 0.01 mL or less, 0.05 mL or less, 0.1 mL or less, 0.5 mL or less, 1 mL or less, 3 mL or less, 5 mL or less, 10 mL or less, 20 mL or less, 25 mL or less, 30 mL or less, 40 mL or less, 50 mL or less, 75 mL or less, 100 mL or less, or 150 mL or less. The volume of such a sample can also range, for example, from 0.01 mL to 150 mL, such as from 0.01 mL to 100 mL, 0.1 mL to 50 mL, 0.1 mL to 10 mL, 0.1 mL to 5 mL, or 0.1 mL to 1 mL. Such a volume can also range, for example, from 0.1 mL to 100 mL, 1 mL to 100 mL, 5 mL to 100 mL, 10 mL to 75 mL, 25 mL to 75 mL, or 40 mL to 60 mL.

Additionally, according to aspects of the methods, the methods include flowing a sample through, e.g., completely through or substantially through, a porous membrane coated with a capture matrix, for example, to concentrate an analyte, for example, to provide a captured sample, according to the subject methods, within a particular time period. Such a time period can be 1 day or less, such as 12 hours or less, such as 6 hours or less, such as 3 hours or less, such as 2 hours or less, such as 1.5 hours or less, such as 1 hour or less, such as 45 min or less, such as 30 min or less, such as 25 min or less, such as 20 min or less, such as 15 min or less, such as 10 min or less, such as 9 min or less, such as 8 min or less, such as 7 min or less, such as 6 min or less, such as 5 min or less, such as 4 min or less, such as 3 min or less, such as 1 min or less. In some versions of the methods, flowing such a sample as described herein and detecting the captured analyte as also described herein can both be performed in such a time period.

In various aspects, the methods also include detecting analyte, e.g., captured analyte, or one or more characteristics thereof, e.g., a concentration and/or identity of an analyte. Such detection can be performed while the analyte is on and/or in the coated membrane and/or on and/or within a housing and/or a container. Such detection can also include generating a signal from the analyte representing one or more characteristics of the analyte and analyzing the signal to recognize the characteristics.

In various embodiments, detecting an analyte can include performing amplification, such as nucleic acid amplification on the analyte. Performing nucleic acid amplification on the analyte can include performing an amplification reaction. As used herein, the phrases “nucleic acid amplification” or “amplification reaction” refers to methods of amplifying DNA, RNA, or modified versions thereof. Nucleic acid amplification includes several techniques, such as an isothermal reaction or a thermocycled reaction. More specifically, nucleic acid amplification includes, but is not limited to, methods such as polymerase chain reaction (PCR), loop-mediated isothermal amplification (LAMP), strand displacement amplification (SDA), recombinase polymerase amplification (RPA), helicase dependent amplification (HDA), multiple displacement amplification (MDA), rolling circle amplification (RCA), and nucleic acid sequence-based amplification (NASBA). The phrase “isothermal amplification” refers to an amplification method that is performed without changing the temperature of the amplification reaction. Protons are released during an amplification reaction: for every deoxynucleotide triphosphate (dNTP) that is added to a single-stranded DNA template during an amplification reaction, one proton (H⁺) is released.

Furthermore, PCR techniques which may be applied according to the subject embodiments are disclosed in the following published US patent applications and International patent applications: US 2008/0166793, WO 08/069884, US 2005/0019792, WO 07/081386, WO 07/081387, WO 07/133710, WO 07/081385, WO 08/063227, US 2007/0195127, WO 07/089541, WO 07030501, US 2007/0052781, WO 06096571, US 2006/0078893, US 2006/0078888, US 2007/0184489, US 2007/0092914, US 2005/0221339, US 2007/0003442, US 2006/0163385, US 2005/0172476, US 2008/0003142, and US 2008/0014589, each of which are incorporated by reference herein in their entirety for all purposes.

The terms “react” or “reaction,” as used herein, refer to a physical, chemical, biochemical, or biological transformation that involves at least one substance, e.g., reactant or reagent and that generally involves (in the case of chemical, biochemical, and biological transformations) the breaking or formation of one or more bonds such as covalent, noncovalent, van der Waals, hydrogen, or ionic bonds. The term includes typical photochemical and electrochemical reactions, typical chemical reactions such as synthetic reactions, neutralization reactions, decomposition reactions, displacement reactions, reduction-oxidation reactions, precipitation, crystallization, combustion reactions, and polymerization reactions, as well as covalent and noncovalent binding, phase change, color change, phase formation, dissolution, light emission, changes of light absorption or emissive properties, temperature change or heat absorption or emission, conformational change, and folding or unfolding of a macromolecule such as a protein.

As such, the methods can include adding a nucleic acid amplification reagent solution to an analyte, whether bound to the capture matrix or released after capture. A nucleic acid amplification preparation solution can be a solution that prepares a biological sample for amplification.

According to some embodiments, a reagent solution includes one or more lysing agent, such as one or more detergent. Such a lysing agent can, for example, include dithiothreitol (DTT), detergents, e.g., Triton X-100, Tween, SDS, dichlorodiphenyltrichloroethane (DDT), chaotropic salts, acids and/or bases, pH buffers, beads, solvents, or any combinations thereof. Such an agent can lyse cells of a biological sample to release nucleic acids therefrom. A reagent solution can also include H₂O and/or one or more buffer.

According to various embodiments, the porous membrane coated with a capture matrix is in a container, e.g., a housing. In such embodiments, the methods can include performing detection and/or amplification of an analyte as described herein in situ while the analyte retained by the coated membrane is within the container and/or housing. As used herein, in situ amplification refers to amplification performed in the same container as the coated membrane. As such, according to the methods, an analyte can be flowed through a porous membrane coated with a capture matrix in a container to concentrate the analyte and then the concentrated analyte can be amplified in situ in the container without eluting the concentrated analyte from the membrane.

Also, in various embodiments, the methods include performing flow-through capture of analyte, e.g., nucleic acids, with a porous membrane coated with a capture matrix. By “flow-through capture” is meant retaining analyte on or in a coated membrane while flowing a sample or a portion, e.g., a portion which does not include or does not substantially include, analyte through the membrane.

The methods described herein can be applied when analyzing samples with low concentrations of analytes, for example rare nucleic acids or proteins, markers and biomarkers of genetic or infectious disease, environmental pollutants, etc. (See e.g., U.S. Pat. No. 7,655,129, incorporated herein by reference in its entirety for all purposes). Another example application includes the analysis of rare cells, such as circulating cancer cells or fetal cells in maternal blood for prenatal diagnostics. Such an approach may be beneficial for rapid early diagnostics of infections by capturing and further analyzing microbial cells in blood, sputum, bone marrow aspirates and other bodily fluids such as urine and cerebral spinal fluid.

According to some embodiments, the device may be used for rapid detection and drug susceptibility screening of bacteria in samples, including complex biological matrices, without pre-incubation.

The subject methods and devices can be used to detect organisms. The term “organism” refers to any organisms or microorganism, including bacteria, yeast, fungi, viruses, protists (protozoan, micro-algae), archaebacteria, and eukaryotes. The term “organism” refers to living matter and viruses including nucleic acid that can be detected and identified by the methods of the disclosure. Organisms include, but are not limited to, bacteria, archaea, prokaryotes, eukaryotes, viruses, protozoa, mycoplasma, fungi, and nematodes. Different organisms can be different strains, different varieties, different species, different genera, different families, different orders, different classes, different phyla, and/or different kingdoms.

In various aspects, the embodiments do not include antibody-coated magnetic beads or rods (e.g., Cell Search, MagSweeper, or RoboSCell technologies), microfluidic posts (e.g., CTC-chip or exosome capture), or microfluidic channel walls, functional capture by unique behaviors including metastatic invasion of collagen adhesion matrices, negative selection by removing all other targets, capture by magnetic, optical, other properties (e.g., by dielectrophoretic field-flow fractionation or photoacoustics), and/or screening all targets visually and collecting those of interest, including by flow cytometry, fiber optic arrays, and/or or laser scanning (Laser-Enabled Analysis and Processing, LEAP™, by Cytellect).

Various embodiments of the subject disclosure enable a multitude of upstream or downstream applications, including combining upstream sample preparation with capture and downstream multi- or single-cell analysis and manipulation. Examples of the types of analysis that can be carried out include, but are not limited to, PCR and other nucleic-acid based tests, immunoassays, staining, including immunostaining, histological staining, and mass-spectrometry. Procedures that can be carried out after isolation include, but are not limited to, cultivation, including cultivation of single cells, pure cultures (one cell type), mixed co-cultures, or spatially-organized co-cultures, stimulus-response assays, including but not limited to antigen, pathogen, or cytokine challenges, receptor binding and chemotaxis assays.

Prior to flowing through the coated membrane, analytes can be selected by size or morphology, for example by filtration. For example, samples can be passed through a filtration device, e.g., a porous membrane coated with a capture matrix, by a process such as aspiration or flow. Filtration devices, such as sieves, retain targets larger than the filtration in the capture area. They can be used to isolate larger targets, or to remove material from smaller targets of interest.

Analytes, e.g., targets, of interest can be captured by their affinity for a capture agent, which can be either specific or non-specific for the target of interest. In certain embodiments, the bulk of the sample is not captured by the device, while the desired targets, such microorganisms, cells, or molecules can be bound and enriched.

The capture matrix comprises a capture agent having the ability to bind molecules of interest specifically or non-specifically or to significantly retard their movement. The capture matrix may be comprised of, for example, a gel formed in and around the support structure, e.g. a membrane. In certain implementations, the gel is a hydrogel comprising the capture agent. Optionally, hydrogel of the capture agent is crosslinked on the membrane. In some versions, the cross-linked hydrogel is not covalently bound to the membrane, but rather remains in proximity to the membrane through other physical forces, e.g., capillary force, ionic bonds or hydrophobic interactions.

A capture matrix can comprise a capture agent, which can include affinity reagents, including antibodies, aptamers, non-specific agents, including for example a hydrophilic patch to which a droplet or cell can stick. Several different capture agents can be included in the same capture matrix.

Analytes of interest can be captured by a unique behavior. For example, cells can be bound to a porous membrane coated with a substance such as a collagen adhesion matrix. Metastatic cells will migrate into the gel, while other cells will not. Other cells can be washed away. In certain implementation, the gel can be dissolved, leaving metastatic cells isolated that can be assayed in situ or moved to another area for analysis.

Capture methods can be combined with downstream analysis and manipulation, including, for example, stimulus-response assays and directed crawling assays. Stimulation-response assays are useful for detection and characterization of cells whose phenotypes are not apparent under resting conditions, for example for the detection of liquid tumors. Captured cells can be stimulated, such as with cytokines, and their response assayed by a set of parallel analyses and manipulations including ELISA for secreted signals including cytokines and proteases, staining for phosphorylation status to determine signaling pathways, PCR, RT-PCR, and culturing. Directional crawling assays may be used to distinguish cells with varying phenotypes. For example, metastatic cells crawl rapidly and directionally when mechanically confined; captured CTCs can be slipped into channels such as long straight ducts in order to assess this behavior.

### Devices

Also provided herein are analyte capture devices, such as flow-through low concentration analyte capture devices. In various embodiments, the devices include a membrane coated with a capture matrix and/or a housing. As used herein, a “porous membrane coated with a capture matrix” is a porous membrane that is coated on a single face, within the pores, or fully encapsulated, with a cohesive matrix comprising a capture agent. The coated membrane retains one substance, e.g., an analyte, more effectively, e.g., substantially more effectively, than another substance, e.g., water and/or buffer, when both of the substances are exposed to the coated membrane and at least one of them is moved at least partially therethrough. For example, a coated membrane, as described herein, upon having a biological sample flowed therethrough will retain an analyte, e.g., nucleic acids, while a remaining analyte-depleted portion of the sample passes through or substantially through the membrane.

Membranes coated with a capture matrix can be configured to capture and thereby concentrate analyte, e.g., concentrate analyte from a first concentration to a second concentration as described herein, from a sample flowed through by any of the amounts of analyte concentration described herein in association with the methods, such as 1000× or more in any of the time amounts described herein, such as in 30 min or less. Such a membrane can also be operatively coupled to the housing, such as attached at the circumferential membrane edges.

By “operatively coupled,” as used herein, is meant connected in a specific way that allows the disclosed devices to operate and/or methods to be carried out effectively in the manner described herein. For example, operatively coupling can include removably coupling or fixedly coupling two or more aspects. Operatively coupling can also include fluidically and/or electrically and/or mateably and/or adhesively coupling two or more components. Also, by “removably coupled,” as used herein, is meant coupled, e.g., physically and/or fluidically and/or electrically coupled, in a manner wherein the two or more coupled components can be un-coupled and then re-coupled repeatedly.

One embodiment of a device according to the subject embodiments includes a cylindrical coated membrane having a membrane core and a coating layer. In some versions, the devices include a housing fully containing the coated membrane therein and including a sample inlet and a sample outlet.

Also, in various embodiments, the housing includes a container, e.g., a cylindrical container, and the coated membrane is positioned within, such as entirely retained between at least two opposing portions of, the container.

Also, as noted above, in some aspects, a coated membrane can include a membrane and a coating layer. In various embodiments, the membrane is a porous structure and includes, e.g., entirely includes, a polymeric material, such as poly-L-lysine and/or nylon, such as a hydroxylated nylon and can include LoProdyne. The matrix coating the membrane can also include, such as entirely include, a gel, such as a hydrogel and/or a fabric, such as cotton. In some versions, the membrane is composed from polypropylene, poly-ethylene glycol (PEG), polyimide, parylene, polycarbonate, cyclic olefin polymer, and/or polymethylmethacrylate, or any combination thereof. In some versions, the coating layer includes, e.g., entirely includes, a cross-linked polysaccharide, such as chitosan. The coating layer can be present on all external surfaces of the membrane, on selected surfaces or only within the pores.

Each of the components of the subject devices, such as the housing, the membrane and/or the coating layer, can be composed of a variety of materials, such as a single material, or a plurality of materials, such as two, three, four, five, or ten or more materials. Each of such components can include one or more flexible materials, such as a layer of flexible material coating a core composed of one or more rigid materials. By “flexible,” as used herein is meant pliable or capable of being bent or flexed repeatedly (e.g., bent or flexed with a force exerted by a human hand or other body part) without damage (e.g., physical deterioration). Such components can also include one or more polymeric materials (e.g., materials having one or more polymers including, for example, plastic and/or rubber and/or foam) and/or metallic materials. Such materials can have characteristics of flexibility and/or high strength (e.g., able to withstand significant force, such as a force exerted on it by use, without breaking and/or resistant to wear) and/or high fatigue resistance (e.g., able to retain its physical properties for long periods of time regardless of the amount of use or environment).

According to the subject embodiments, materials of interest that any of the device components described herein can be composed of include, but are not limited to: polymeric materials, e.g., photopolymer materials such as Veroclear, and TangoPlus, and/or plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals and metal alloys, e.g., titanium, chromium, stainless steel, etc., and the like. The materials can be transparent or semi-transparent such that a device user can observe a biological sample and/or a preparation solution throughout device operation. By utilizing translucent materials, fluids are visible as they are transported through the device, providing visual feedback during operation.

Also, in various embodiments, the porous membrane is cylindrical and/or as such, is symmetrical about an axis, e.g., an axis of symmetry. In some embodiments, a membrane for use in the methods described herein can be cylindrical, and can have a consistent circular, oblong, rectangular, triangular cross-sectional shape and/or diameter and/or circumference along its length. A membrane can extend along a length, such as a length from a first end to a second end opposite the first end. A membrane can also have edges defined by and contacting an edge, e.g., an interior edge of the housing or an opening within the housing and/or container.

A membrane suitable for coating with a capture matrix can have pore dimensions, e.g., pore radius, and/or length. In various embodiments, a pore dimension, e.g., pore radius, ranges from 0.01 μm to 5 μm, such as 0.1 μm to 3 μm, such as 0.1 μm to 1 μm. A membrane suitable for coating can have a pore length (Sm) and/or membrane thickness, ranging from 0.1 μm to 4000 μm, such as from 0.2 μm to 3500 μm, or 0.3 μm to 3000 μm, or from 0.1 μm to 100 μm, such as 0.5 μm to 50 μm, such as 0.5 μm to 20 μm. A membrane suitable for coating can have a pore length (Sm) and/or membrane thickness, ranging from 10 μm to 10000 μm, 500 μm to 2000 μm, or 100 μm to 1000 μm. The membrane can have a radius (Rₘ), ranging from 0.1 mm to 10 mm, such as 0.1 mm to 5 mm, such as 0.1 mm to 3 mm. A membrane can also have a radius of 5 cm or less, such as 3 cm or less, such as 2 cm or less, such as 1 cm or less, such as 0.5 cm or less, such as 0.1 cm or less, such as 0.01 cm or less, such as 0.001 cm or less, or 3 mm or less, 2 mm or less, or 1 mm or less. A membrane suitable for coating with a capture matrix can also have a pore length of 4000 μm or less, such as 3500 μm or less, such as 3000 μm or less, such as 2000 μm or less, such as 1000 μm or less, such as 500 μm or less, such as 250 μm or less, such as 200 μm or less, such as 160 μm or less, such as 150 μm or less, such as 100 μm or less, such as, such as 10 μm or less, such as 1 μm or less such as 0.5 μm or less. Also, a membrane can be cylindrical and can have a diameter or radius, e.g., a cross-sectional diameter or radius, ranging from 1 mm to 10 cm, such as 1 mm to 5 cm, such as 1 mm to 1 cm, such as 1 mm to 10 mm, or from 1 mm to 5 mm, such as 1 mm to 3 cm. The pores of such a membrane can have a diameter or radius (Rₚ), e.g., a cross-sectional diameter or radius, ranging from 0.1 μm to 100 μm, such as 0.1 μm to 50 μm, such as 0.1 μm to 20 μm, such as 0.1 μm to 1 μm. The pores of such a membrane can have a cross-sectional radius of 10 μm or less, such as 5 μm or less, such as 1 μm or less, such as 0.9 μm or less, or of 0.8 μm or less, 0.75 μm or less, 0.6 μm or less, or 0.7 μm or 0.5 μm or less.

### Utility

Detecting nucleic acids (NAs) at zeptomolar concentrations, such as concentrations of a few, e.g., 100 or less, 50 or less, 10 or less, 1 or less, molecules per milliliter, can require expensive equipment and lengthy processing times to isolate and concentrate the NAs into a volume that is amenable to amplification processes, such as PCR or LAMP. Shortening the time required to concentrate NAs and integrating this procedure with amplification on-device, such as by the methods disclosed herein is important to a number of analytical fields, including environmental monitoring and clinical diagnostics. For example, pathogens in aqueous environmental samples are frequently present at or below zeptomolar concentrations (˜1000 microorganisms per liter), requiring laborious filtration and concentration procedures before detection is possible. 6, 7. In many clinical applications, including minimal residual diseases (8) and latent Hepatitis C viral (HCV) or HIV infections, target NAs are also present at <10 molecules/mL. 9, 10. Blood bank donations can be pooled before screening, so targets can be diluted by several orders of magnitude before being screened for pathogens, generating a sample where ultra-sensitive detection is critical. 11, 12. Each of these examples requires the processing of large volumes (mLs) of extremely dilute samples, and therefore the ability to concentrate NAs on the order of 1000× to reach PCR-suitable volumes (μLs). Additionally, the entire concentration process should be done within minutes and not rely on expensive equipment to be directly applicable to limited-resource settings (LRS) and at the point-of-care (POC). 13, 14. Microfluidic point-of-care (POC) devices have been designed to address these needs, but they are not able to detect NAs present in zeptomolar concentrations in short time frames because they require slow flow rates and/or they are unable to handle milliliter-scale volumes.

Additionally, commercial systems for the purification and concentration of nucleic acids can involve solid phase extraction (SPE), which uses chaotropic agents to control the absorption and release of NAs on silica. 15, 16. While this method is widely used, most available protocols require centralized laboratories for centrifuging samples or manipulating beads. 17. NA precipitation (18) methods are also commonly used to extract and concentrate NAs from clinical and environmental samples; however these methods are laborious and involve the use of hazardous reagents. 19. These methods are challenging to deploy for LRS, where instrumentation is limited, or for use at the POC, where diagnostics must be rapid and require minimal sample handling. 17. As such, several charge-based methods have been developed, which can include a charged polymer matrix including chitosan, poly-L-lysine, and so on for NA capture. 20-24. To increase sensitivity, these and other systems concentrate NAs and then either elute before amplification (20, 21, 23, 24) or perform amplification in situ. 22, 25-28. While these methods have some advantages over more common solid-phase extraction methods, processing time and lowest detectable concentration are still limited by their inability to handle large sample volumes (>1 mL) (25-27, 30) and/or their slow processing rates, which range from μL/min to μL/hr. 17, 20, 21, 23, 31, 32. Thus, current methods—whether commercialized or from literature—lack the required combination of sensitivity, speed and ease of implementation, leaving a gap in the current NA detection workflow.

As set forth herein, the methods of applying a flow-through capture membrane that effectively captures NAs with high sensitivity in a short time period were theoretically investigated and experimentally applied. For example, according to the subject methods for nucleic acid detection, ˜10 molecules, e.g., 50 or fewer, 25 or fewer, or 10 or fewer molecules, of DNA 50 mL or less of sample, e.g., 25 mL, or 10 mL or 1 mL, into a 2 mm radius capture membrane can be concentrated. The membrane is also compatible with in situ amplification, which, by eliminating an elution step enables high sensitivity and facile device integration.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Here, it was hypothesized that pressure-driven flow and capture in a porous matrix could facilitate the handling of large samples, while retaining many of the characteristics needed for both LRS and POC. This approach was analyzed theoretically and experimentally as set forth herein to determine a regime in which rapid, convection-driven capture is possible. Using a theoretical framework to predict capture efficiency as a function of flow-through conditions, it was determined the parameters necessary for a detection matrix to capture a few nucleic acid molecules (<10) from several mLs of volume in short times (<10 minutes). The predictions were tested experimentally with respect to capture efficiency, lowest detectable concentration, processing time, and total sample volume. Furthermore, it was demonstrated that the membrane and capture matrix are compatible with direct amplification, eliminating the need for an elution step. The ability to amplify in situ makes this approach amenable to integration into sample-to-answer devices, and preserves the high concentration factors achieved during capture by preventing loss of target to the capture matrix during elution.

### I. CAPTURE SIMULATIONS

The fraction of nucleic acid molecules captured in a membrane pore compared to the amount flowed through (capture efficiency) was simulated at steady-state using the Transport of Diluted Species module of Comsol Multiphysics (version 4.4). A description of the model geometry, transport parameters, kinetics, boundary conditions, mesh, and calculations performed is provided in further detail below.

### II. CHITOSAN HYDROGEL COATED MEMBRANE FABRICATION

A nylon membrane (LoProdyne LPNNG810S, Pall Corp., New York City, N.Y.) was used as a porous matrix support. To prepare hydrogel coated membranes, a 0.5% (w/v) solution of chitosan (TCI OBR6I) was prepared in 150 mM HCl. A 25% (v/v) solution of glutaraldehyde was added to this solution to a final concentration of 4 mM. The solution was rapidly mixed, and added to the LoProdyne membrane in excess. The saturated membranes were then spun on a Laurel WS-400-6NNP/Lite spin coater at 500 rpm for 5 s with an acceleration setting of 410, followed by 15 s at 2000 rpm with an acceleration setting of 820. Membranes were allowed to crosslink for 2 h in air, washed 3 times with NF water, and dried under vacuum.

### III. CAPTURE AND IN SITU AMPLIFICATION

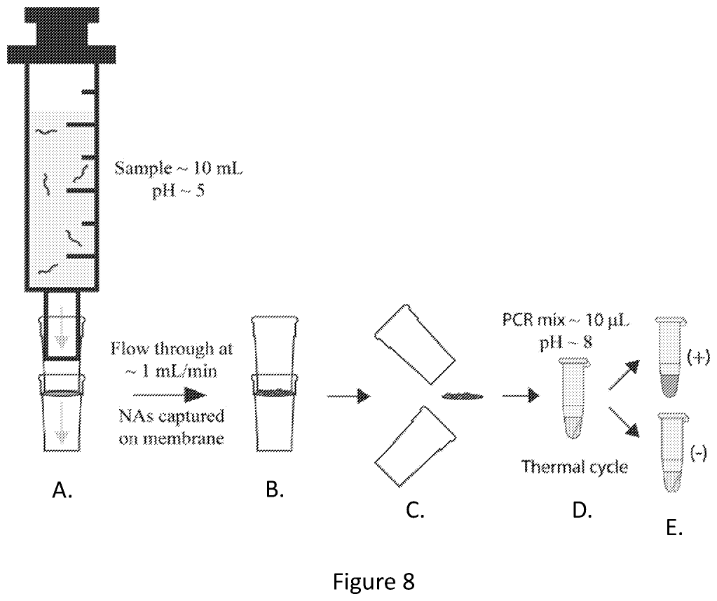

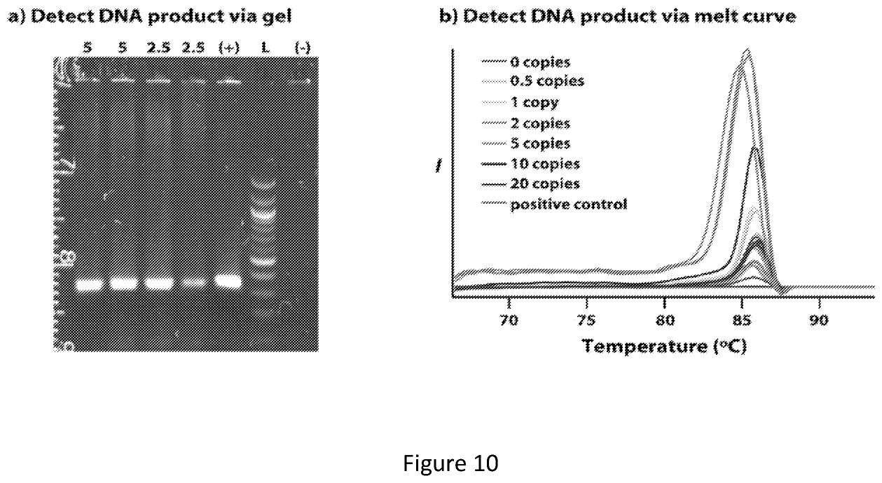

λ-phage DNA stocks were quantified via digital PCR. 33. This DNA was spiked into varying volumes of 10 mM MES buffer (pH ˜5) to create concentrations ranging from 0.2 to 20 copies/mL (Table S-4). The solutions were flowed through 4 mm diameter chitosan-coated nylon membranes at a flow rate of 1 mL/min using syringes and luer locks (FIGS. 8A-E), followed twice by 100 μL MES buffer. The membranes were then removed from the syringe/luer lock system, placed in an Ilumina Eco™ well plate, and 5-10 μL of PCR mix was added to each membrane. The well plate was inserted into an Ilumina Eco™ real time PCR system (EC-101-1001, Ilumina, San Diego, Calif.) and thermal cycled; correct λ-phage product was verified with a gel and melt curve analysis (FIGS. 10A and 10B).

The PCR mixture used for amplification of λ-phage DNA on the chitosan-coated nylon membranes contained the following: 5 μL 2×SsoFast Evagreen SuperMix (BioRad, Hercules, Calif.), 1 μL of BSA (20 mg/mL), 2 μL of 10 ng/uL salmon sperm DNA (Invitrogen), 1 μL of 5 μM primers (Table S-7), and 1 μL of NF water. The PCR amplification was performed with an initial 95° C. step for 3 min and then followed by 40 cycles of: (i) 20 s at 95° C., (ii) 20 s at 62° C., (iii) 15 s at 72° C.

In various aspects, human blood plasma was also processed. λ-phage DNA stocks were quantified via digital PCR. 33. This DNA was spiked into varying volumes of lysed human blood plasma to create concentrations ranging from 2 to 270 copies/mL (Table S-6). Human blood plasma was lysed as follows: 1 mL of “acidification buffer” is made by adding 100 μL 1M sodium acetate to 900 μL acetic acid; then, 125 μL 20 mg/mL proteinase K, 50 μL of 10× Thermopol Reaction buffer, 825 μL of nuclease-free water, and 200 μL of acidification buffer are added for every 1 mL of plasma. The mixture is then incubated at 55° C. for 180 minutes and the lysed plasma is pre-filtered with a 5 μm pore size sterile filter, followed by a 0.45 μm pore size sterile filter. λ DNA was added to various volumes of lysed plasma (see Table S-6) and the resulting solution was flowed through the chitosan membrane at ˜1 mL/min using syringes and luer locks (FIGS. 8A-E), followed twice by 100 μL MES buffer. The membranes were then removed from the syringe/luer lock system, placed in an Ilumina Eco™ well plate, and 5-10 μL of PCR mix was added to each membrane. The well plate was inserted into an Ilumina Eco™ real time PCR system (EC-101-1001, Ilumina, San Diego, Calif.) and thermal cycled; correct k-phage product was verified with a gel and melt curve analysis (FIGS. 10A and 10B).

The PCR mixture used for amplification of λ-phage DNA on the chitosan-coated nylon membranes contained the aspects indicated above in relation to the buffer capture methodology. The PCR amplification was performed with an initial 95° C. step for 3 min and then followed by 40 cycles of: (i) 20 s at 95° C., (ii) 20 s at 62° C., (iii) 15 s at 72° C.

### V. THEORETICAL ANALYSIS

To predict a regime that would enable rapid flow-through capture of nucleic acids present at low concentrations, a theoretical model was developed that takes into account the convection, diffusion, and adsorption of nucleic acid molecules onto a capture matrix layered over and within a porous matrix (FIG. 1A and S-I). The parameters governing capture dynamics are superficial velocity U [m/s], pore radius Rₚ [m], membrane radius Rₘ [m], membrane thickness (or, equivalently, pore length) δₘ [m], diffusivity of nucleic acid molecules³⁴ D [m²/s], association rate constant³⁵ kₒₙ [m³/(mol·s)], surface concentration of the capture agent γ [mol/m²], and mass transfer coefficient k_{c} [m/s]. Instead of analyzing every relevant parameter individually, the parameters were condensed into two dimensionless numbers:^{36,37} Damkohler (Da) and Péclet (Pe). Da characterizes the balance between adsorption rate and transport rate (Eq. 1) while Pe characterizes the balance between convection rate and diffusion rate (Eq. 2).

$$\begin{matrix}D a = \frac{adsorption rate}{transport rate} = \frac{{k}_{o n} γ}{{k}_{c}} , {k}_{c} = 1.62 {( \frac{{UD}^{2}}{2 {δ}_{m} {R}_{p}} )}^{\frac{1}{3}} & ( 1 ) \\ Pe = \frac{convection rate}{diffusion rate} = \frac{U / {δ}_{m}}{D / {R}_{p}^{2}} & ( 2 )\end{matrix}$$

Da>1 indicates that the rate of DNA binding to the capture agent is faster than the rate of DNA transport to the pore wall; Pe<1 means the rate at which molecules diffuse to the pore wall is faster than the rate at which they are convected through the pore. To capture dilute nucleic acids from large volumes in short times, two conditions must be met: i) efficient capture (Da>>1), and ii) fast flow rates (Q˜1 mL/min) while maintaining Pe<1.

Capture efficiency is a factor of binding kinetics (time for the nucleic acid molecule to bind to the capture agent) and transport (time for the nucleic acid molecule to travel from the bulk solution to the pore wall coated with capture matrix). High capture efficiency occurs when the transport rate is slower than the binding reaction rate (i.e., Da>>1), which can occur with fast reactions or slow transport. Many passive capture processes—such as wicking through a porous matrix or mixing with beads—rely on slow transport rates to achieve high Da. These processes capture efficiently at small length scales in microliter volumes; (20-22, 32) however, for milliliter volumes and large length scales, passive capture processes would require impractical amounts of capture agent or time for Da to be greater than 1. A fast binding reaction with diffusion-limited kinetics would enable higher transport rates (and thus faster flow rates) without adversely affecting capture efficiency. Electrostatic binding and silica adsorption in the presence of Ca2+ are examples of diffusion-limited chemical reactions (38, 39) that would maintain high Da without relying on slow transport rates to ensure efficient capture. The simulations show that when a capture matrix coated on a pore wall has fast binding kinetics, Da>10 ensures >95% capture of nucleic acids flowing through the pore (FIG. 1B and S-I). To scale up efficient capture processes to larger volumes, the mass transport rate can be increased. One way to increase mass transport rate is actively forcing fluid through a porous matrix, (40) which is well established in membrane chromatography. 41, 42. However, flow-through capture has not been analyzed theoretically nor tested experimentally for rapid capture and detection of zeptomolar nucleic acids.

In general, high flow rates increase the transport rate, decrease Da, and thus reduce capture efficiency. However, the transport rate can be maintained below the adsorption rate (keeping Da>>1) by manipulating other transport parameters, thus counteracting the high flow rate. These transport parameters can be analyzed together by simulating the capture efficiency as a function of Pe (S-I): simulations show that keeping Pe<1 ensures >90% capture efficiency (FIG. 1C). To achieve a high convection rate and maintain Pe<1, a relatively high diffusion rate is required, which ensures that the molecules don't leave the pore before having a chance to diffuse to the wall and bind. To maintain this balance of a high convection rate with an even higher diffusion rate, the membrane radius, pore radius, and membrane thickness can be adjusted. Setting Pe<1 in Eq. 2 provides the following constraint on flow rate through the membrane (Q) as a function of δm, Rm, and Rp, where ϕ represents the porosity of the membrane (see S-II for derivation).

$$Q < \frac{πϕ D {δ}_{m} {R}_{m}^{2}}{{R}_{p}^{2}} \qquad ( 3 )$$

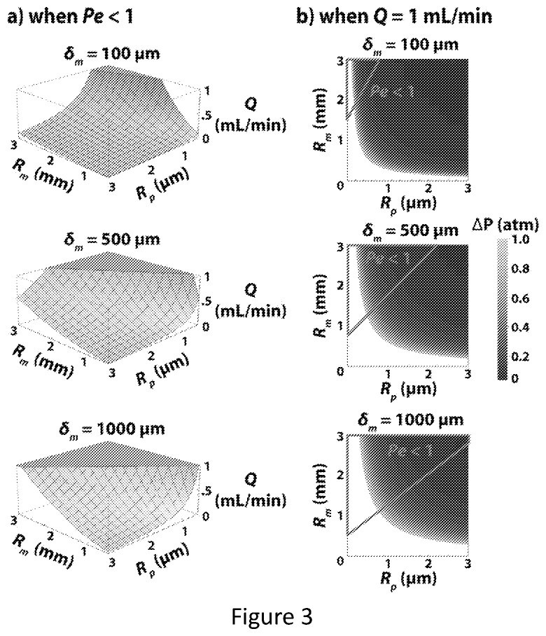

Plotting Eq. 3 at different membrane thicknesses explores the relationship of these parameters (FIG. 3A); trends favoring Pe<1 and flow rates >1 mL/min are decreasing pore radius, increasing membrane radius, and increasing membrane thickness. Decreasing the pore size enables faster diffusion rates and lower Pe, but it also increases the resistance to flow. FIG. 3B considers this tradeoff, showing the pressure drop required for a sample to flow through the membrane at 1 mL/min at different membrane and pore radii. The overlap of the green triangles (Pe<1) with red color (ΔP<1 atm) represents represents an ideal combination of parameters wherein Pe is low enough and a reasonable pressure drop is achieved to flow at 1 mL/min.

### VI. EXPERIMENTAL ANALYSIS

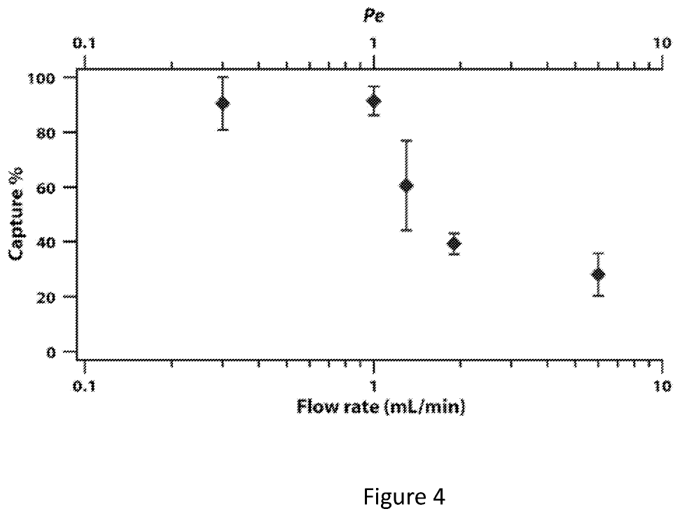

Based on such predictions, an appropriate experimental system was chosen to evaluate the ability of a flow-through matrix to rapidly capture zeptomolar concentrations of nucleic acids. This matrix should be compatible with in situ amplification, so glass fiber, silica, and other common capture materials that inhibit amplification reactions were not considered.^{43,44} Nylon membranes do not prevent nucleic acid amplification and can be purchased in various pore sizes and thicknesses. The membrane thickness for a LoProdyne nylon membrane from Pall Corporation ranges from 127.0-190.5 μm (see “Chitosan Membrane Fabrication” section); at this thickness, a membrane radius of 2 mm is flexible and easily placed in a well plate for nucleic acid amplification. For a membrane thickness of 160 μm, flow rate of 1 mL/min, and membrane radius of 2 mm, Eq. 3 predicts that pore radii less than 0.76 μm would maintain Pe<1. Therefore, LoProdyne membranes were chosen with a pore radius of 0.6 μm; coating the membrane pores with a capture matrix makes the pore size even smaller, ensuring that the application was well below the 0.76 μm requirement. As described, the capture agent must have diffusion-limited kinetics. Because electrostatic binding is very fast and can easily be used for nucleic acid capture utilizing a cationic polymer to attract the negatively charged phosphate backbone of DNA, chitosan was chosen as the capture agent, which has previously been used for NA capture. 20-24. Chitosan is an inexpensive biocompatible polymer with amine groups on its backbone that become positively-charged when the pH is below 6.3. 21, 45. Chitosan was coated onto the nylon membrane as described in “Chitosan Membrane Fabrication” section. To verify that coating the membrane with chitosan does not reduce the pore size such that the pressure drop becomes untenable (FIG. 3B), the capture efficiency was measured at different flow rates. This experiment showed that the chitosan-coated nylon membrane captures >90% of nucleic acids when solution is flowed through at 1 mL/min (see FIG. 4).

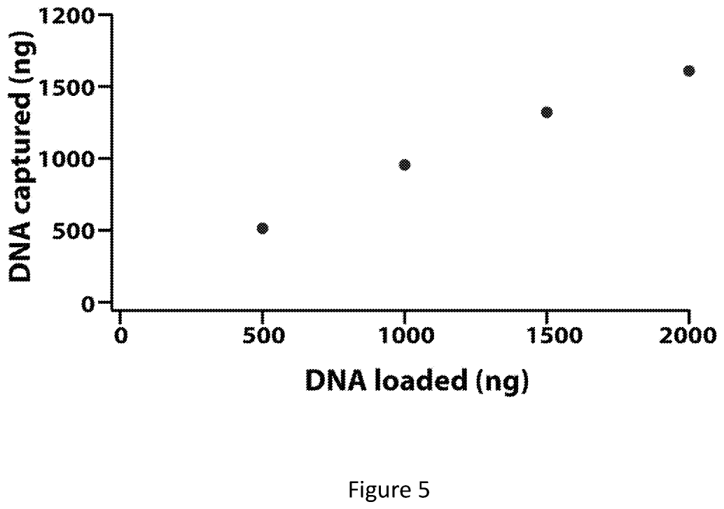

To test the predictions from the analysis, the DNA binding capacity of chitosan membranes was measured and the capture efficiency as a function of Pe was evaluated. It was found that chitosan-coated nylon membranes have a capacity of 1000 ng or more (FIG. 5). Even though the provided application is for low amounts of nucleic acid, this matrix can also capture large amounts of genetic material for other applications. It was also confirmed that the chitosan membranes capture efficiently over a range of Pe, with >90% capture of DNA when Pe<1 (FIG. 4).

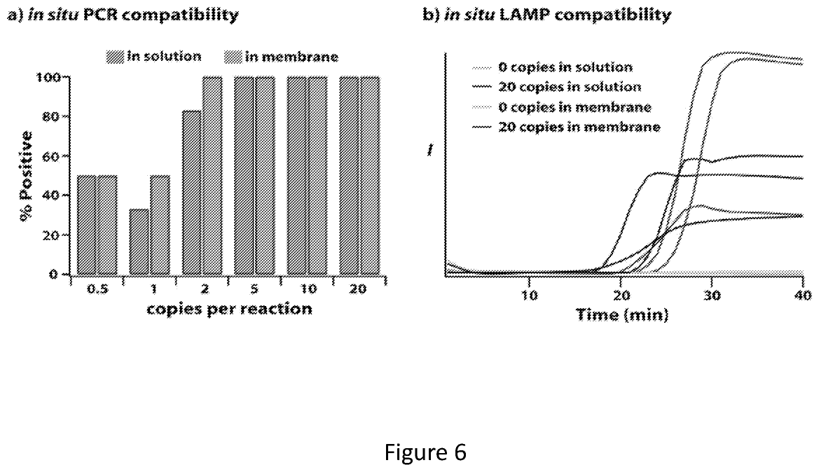

Next, it was tested whether in situ amplification would work with the nylon membrane that had been coated with chitosan. Serial dilutions of DNA were added to the membrane, then submerged in amplification mix and amplified DNA via PCR. The chitosan membrane was compatible with in situ PCR amplification down to ˜2 copies per reaction (FIG. 6A). The chitosan membrane compatibility with in situ LAMP was also tested and showed successful amplification at 20 copies per reaction (FIG. 6B⁴⁶).

The final step was to use chitosan's charge-switch capability to couple rapid capture with direct amplification without eluting the nucleic acids. A sample flows through the chitosan-coated membrane at pH ˜5 and the negatively-charged phosphate backbone of DNA will electrostatically bind to the positively-charged amine groups on the chitosan. Following capture of NAs, the addition of amplification mix at pH ˜8 deprotonates the amine groups and releases the captured nucleic acids for amplification (FIGS. 4A and 4B).

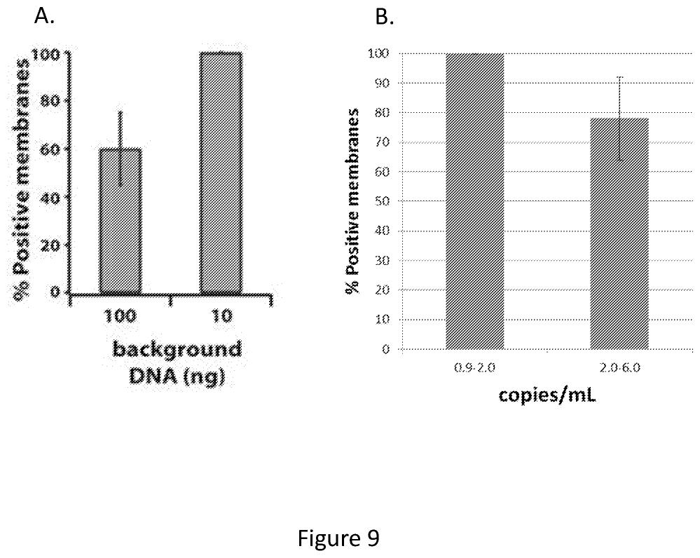

This idea was then tested (combining rapid capture and in situ amplification via charge-switch) at ultra-low concentrations (˜1 copy/mL). Various amounts of λ DNA were spiked into volumes ranging from 1 to 50 mL (Table S-4 and Table S-5) and the solution flowed through a 2 mm radius chitosan-coated membrane at ˜1 mL/min. After capture, the amplification was performed in situ with small volumes of PCR reagents (5-10 μL), as opposed to the traditional method of eluting from a capture matrix and using larger volumes of PCR reagents. DNA product was detected after thermal cycling using EvaGreen dye (see SI-V for details). This methodology detected a DNA target at concentrations as low as 0.5 copies/mL from as many as 50 mL (FIG. 9A). Compiling data from replicate experiments run on different days, pre-concentration using the chitosan-coated membrane allowed detection down to 0.9 copy/mL over 91% of the time.

This idea was also tested for its ability to capture and amplify nucleic acids from a biological solution such as human blood plasma. Various amounts of λ DNA were spiked into volumes of lysed plasma ranging from 2 to 20 mL (Table S-6) and the solution flowed through a 2 mm radius chitosan-coated membrane at ˜1 mL/min. After capture, the amplification was performed in situ with small volumes of PCR reagents (5-10 μL), as opposed to the traditional method of eluting from a capture matrix and using larger volumes of PCR reagents. DNA product was detected after thermal cycling using EvaGreen dye (see XI for details). This methodology detected a DNA target at concentrations as low as 5 copies/mL from as many as 2 mL of plasma (Table S-6). Compiling data from replicate experiments run on different days, pre-concentration using the chitosan-coated membrane allowed detection down to 10-20 copies/mL of plasma over 64% of the time.

### VII. FLOW-THROUGH CAPTURE SIMULATIONS

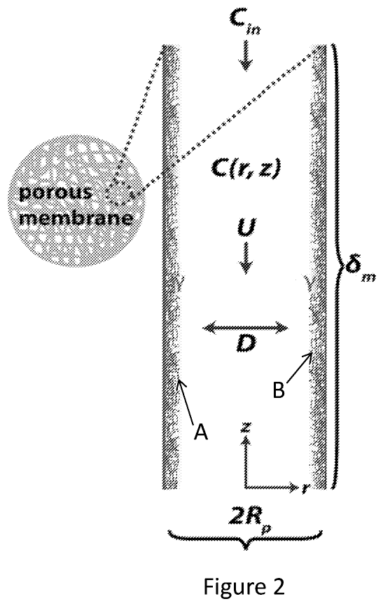

The fraction of nucleic acid molecules captured in a membrane pore compared to the amount flowed through (capture efficiency) is a function of pore geometry, flow parameters, and adsorption kinetics (FIG. 2). The concentration of nucleic acids at any position in the pore, C(r, z), was simulated at steady-state using the Transport of Diluted Species module of Comsol Multiphysics (version 4.4) with the parameters listed in Table S-1. To generate the data for FIGS. 1B and 1C., a parametric sweep was performed with various values of kₒₙ·γ, U, Rₚ, and δₘ (Table S-2 and Table S-3). Then, the inlet flux (Jᵢₙ=J|_{z=δm}) and outlet flux (Jₒᵤₜ=J|_{z=0}) were evaluated and used in Eq. S-1 to calculate capture efficiency.

$$Capture \% = 1 - \frac{{J}_{out}}{{J}_{i n}} \qquad ( S \text{-} 1 )$$

TABLE S-1
Parameters used in the flow-through capture simulations.

| Parameter | Description | Value | |
|---|---|---|---|
| Rₚ | Pore radius | 0.56-17.78 | μm |
| δₘ | Pore length (thickness of | 0.316-3162 | μm |
| | membrane) | | |
| U | Flow velocity | 0.118-1000 | mm/s |
| D | Diffusivity of nucleic acid | 10 | μm² · s⁻¹ |
| | molecule | | |
| kₒₙ | Nucleic acid binding rate | 10⁶ | L · mol⁻¹ · s⁻¹ |
| | constant | | |
| γ | Surface concentration of | 10⁻⁷ | mol · m⁻² |
| | capture agent | | |
| Cᵢₙ | Inlet concentration of | 1 | μM |
| | nucleic acids | | |

TABLE S-2
The product of kₒₙ · γ was varied to generate Capture %
as a function of Damköhler number (Da) (FIG. 1B). Rₚ (1 μm), δₘ
(100 μm), U (2 mm/s), D (10 μm² · s⁻¹),
and Cᵢₙ (1 μM) were held constant.

| kₒₙ · γ | k_{c} | | Jᵢₙ | Jₒᵤₜ | |
|---|---|---|---|---|---|
| (m/s) | (m/s) | Da | (mol/s) | (mol/s) | Capture % |
| 1.00E−07 | 1.62E−05 | 0.01 | −3.92E−18 | −3.88E−18 | 1.0 |
| 2.15E−07 | 1.62E−05 | 0.01 | −3.92E−18 | −3.83E−18 | 2.1 |
| 4.64E−07 | 1.62E−05 | 0.03 | −3.92E−18 | −3.74E−18 | 4.5 |
| 1.00E−06 | 1.62E−05 | 0.06 | −3.92E−18 | −3.55E−18 | 9.3 |
| 2.15E−06 | 1.62E−05 | 0.13 | −3.92E−18 | −3.19E−18 | 18.5 |
| 4.64E−06 | 1.62E−05 | 0.29 | −3.92E−18 | −2.58E−18 | 34.2 |
| 1.00E−05 | 1.62E−05 | 0.62 | −3.92E−18 | −1.75E−18 | 55.2 |
| 2.15E−05 | 1.62E−05 | 1.33 | −3.92E−18 | −9.77E−19 | 75.0 |
| 4.64E−05 | 1.62E−05 | 2.87 | −3.92E−18 | −5.03E−19 | 87.2 |
| 1.00E−04 | 1.62E−05 | 6.17 | −3.92E−18 | −2.94E−19 | 92.5 |
| 2.15E−04 | 1.62E−05 | 13.3 | −3.92E−18 | −2.11E−19 | 94.6 |
| 4.64E−04 | 1.62E−05 | 28.7 | −3.92E−18 | −1.78E−19 | 95.5 |
| 1.00E−03 | 1.62E−05 | 61.7 | −3.92E−18 | −1.63E−19 | 95.8 |
| 2.15E−03 | 1.62E−05 | 133 | −3.92E−18 | −1.57E−19 | 96.0 |

TABLE S-3
U, δₘ, or Rₚ was varied to generate Capture % as a function of
Péclet number (Pe) (FIG. 1C). Cᵢₙ (1 μM), kₒₙ · γ (10⁻⁴ m/s),
and D (10 μm² · s⁻¹) were held constant.

| U | δₘ | Rₚ | | Jᵢₙ | Jₒᵤₜ | |
|---|---|---|---|---|---|---|
| (m/s) | (μm) | (μm) | Pe | (mol/s) | (mol/s) | Capture % |
| 1.18E−04 | 100 | 1 | 0.12 | −2.46E−19 | −2.32E−36 | 100.0 |
| 2.68E−04 | 100 | 1 | 0.27 | −5.32E−19 | −1.00E−26 | 100.0 |
| 6.11E−04 | 100 | 1 | 0.61 | −1.20E−18 | −4.11E−22 | 100.0 |
| 1.39E−03 | 100 | 1 | 1.39 | −2.72E−18 | −7.21E−20 | 97.4 |
| 3.16E−03 | 100 | 1 | 3.16 | −6.19E−18 | −1.11E−18 | 82.0 |
| 7.20E−03 | 100 | 1 | 7.20 | −1.41E−17 | −5.92E−18 | 58.0 |
| 1.64E−02 | 100 | 1 | 16.4 | −3.21E−17 | −2.02E−17 | 37.0 |
| 3.73E−02 | 100 | 1 | 37.3 | −7.30E−17 | −5.70E−17 | 22.0 |
| 8.48E−02 | 100 | 1 | 84.8 | −1.66E−16 | −1.46E−16 | 12.3 |
| 1.93E−01 | 100 | 1 | 193 | −3.78E−16 | −3.54E−16 | 6.5 |
| 4.39E−01 | 100 | 1 | 439 | −8.60E−16 | −8.32E−16 | 3.2 |
| 1.00E+00 | 100 | 1 | 1000 | −1.96E−15 | −1.93E−15 | 1.6 |
| 2.00E−03 | 3162 | 1 | 0.06 | −3.90E−18 | 8.30E−39 | 100.0 |
| 2.00E−03 | 1000 | 1 | 0.20 | −3.90E−18 | −1.15E−28 | 100.0 |
| 2.00E−03 | 316 | 1 | 0.63 | −3.90E−18 | −1.72E−21 | 100.0 |
| 2.00E−03 | 100 | 1 | 2.00 | −3.90E−18 | −2.90E−19 | 92.6 |
| 2.00E−03 | 31.6 | 1 | 6.32 | −3.90E−18 | −1.50E−18 | 61.5 |
| 2.00E−03 | 10.0 | 1 | 20.0 | −3.90E−18 | −2.65E−18 | 32.1 |
| 2.00E−03 | 3.16 | 1 | 63.2 | −3.90E−18 | −3.30E−18 | 15.4 |
| 2.00E−03 | 1.00 | 1 | 200 | −3.90E−18 | −3.65E−18 | 6.4 |
| 2.00E−03 | 0.316 | 1 | 632 | −3.90E−18 | −3.80E−18 | 2.6 |
| 2.00E−03 | 100 | 0.56 | 0.63 | −1.23E−18 | −1.60E−21 | 99.9 |
| 2.00E−03 | 100 | 1.00 | 2.00 | −3.90E−18 | −2.90E−19 | 92.6 |
| 2.00E−03 | 100 | 1.78 | 6.32 | −1.23E−17 | −4.20E−18 | 65.9 |
| 2.00E−03 | 100 | 3.16 | 20.0 | −3.90E−17 | −2.30E−17 | 41.0 |
| 2.00E−03 | 100 | 5.62 | 63.2 | −1.23E−16 | −9.40E−17 | 23.6 |
| 2.00E−03 | 100 | 10.00 | 200 | −3.90E−16 | −3.40E−16 | 12.8 |
| 2.00E−03 | 100 | 17.78 | 632 | −1.23E−15 | −1.15E−15 | 6.5 |

A. Geometry:

The model was assembled using a cylindrical geometry drawn in 2D axially symmetric space, with r as the radial component and z the axial component (FIG. 2). The radius of the cylinder (Rₚ) varied from 0.56 μm to 17.78 μm; the length of the cylinder (δₘ) varied from 0.316 μm to 3162 μm (Table S-3).

B. Transport:

In a porous matrix, fluid flow can be approximated with a uniform velocity (U) independent of radius⁴⁷. The flow velocity varied from 1.18·10⁻⁴ m/s to 1 m/s (Table S-3). The top boundary of the cylinder (z=δₘ) was an inlet and the bottom boundary (z=0) was an outlet. The diffusion coefficient used was for DNA², 10⁻¹¹ m²/s.

C. Kinetics:

The binding rate between nucleic acids and the capture agent was assumed to be second order with respect to nucleic acid concentration and capture agent surface concentration. The surface concentration of capture agent (γ) was assumed to be in excess (and therefore unchanging during the course of the adsorption reaction) and estimated it to be 10⁻⁷ mol/m (48).

With a kinetic rate constant estimated from nucleic acid-cationic polymer kinetics⁴⁹, the adsorption rate occurring at the pore wall is shown in Eq. S-2.

R_{ads}=kₒₙ·y·C(Rₚ,z) (S-2)

D. Boundary Conditions:

The inlet concentration of nucleic acid molecules (Cᵢₙ=10⁻⁶ mol/L) represents a normal nucleic acid concentration in human blood plasma⁵⁰. Axial symmetry was imposed at r=0, and a flux boundary condition (Eq. S-3) was imposed at r=Rₚ to represent the adsorption of nucleic acid molecules to the surface of the pore wall.

$${R}_{ads} = D \frac{\partial C ( r , z )}{\partial r} \vert r = {R}_{p} \qquad ( S \text{-} 3 )$$

E. Mesh and Solver Settings:

The geometry was meshed using a Free Triangular mesh with a maximum element size of 0.0525 μm. The Direct Stationary Solver (PARDISO) was used with a nested dissection multithreaded preordering algorithm and an auto scheduling method.

### VIII. CALCULATIONS OF MEMBRANE RADIUS, PORE RADIUS AND MEMBRANE THICKNESS

The number of pores in a membrane (nₚ) can be calculated from the porosity (ϕ) as in Eq. S-4.

$$∅ = \frac{{N}_{p} π {R}_{p}^{2}}{π {R}_{m}^{2}} \to {n}_{p} = \frac{∅ {R}_{m}^{2}}{{R}_{p}^{2}} \qquad ( S \text{-} 4 )$$

The flow rate through the entire membrane (Q) is the flow rate through each pore (Qₚ) multiplied by the number of pores (Q=nₚQₚ). Using Eq. S-4 for nₚ and solving for Qₚ gives the following:

$${Q}_{p} = \frac{Q {R}_{p}^{2}}{∅ {R}_{m}^{2}} \qquad ( S \text{-} 5 )$$

Eq. S-6 results from plugging Eqn S-5 into the relationship between pore flow rate and flow velocity (Qₚ=UπRₚ²).

$$U = \frac{{Q}_{p}}{π {R}_{p}^{2}} = \frac{Q}{π ∅ {R}_{m}^{2}} \qquad ( S \text{-} 6 )$$

Then, using Eq. S-6 in Eq. 2 and setting the condition that Pe<1 yields Eq. S-7.

$$P e = \frac{U {R}_{p}^{2}}{D {δ}_{m}} = \frac{Q {R}_{p}^{2}}{π ∅ {R}_{m}^{2} D {δ}_{m}} < 1 \qquad ( S \text{-} 7 )$$

Solving Eq. S-7 for Q yields Eq. 3. ϕ=0.6 and D=10⁻¹¹ m²/s were assumed for all calculations.

To calculate the pressure drop as a function of pore radius (Rₚ) and membrane radius (Rₘ), Pouiselle flow was assumed (Eq. S-8). Flow rate through the pore (Qₚ) was replaced with flow rate through the entire membrane (Q) using Eq. S-5. Q (1 mL/min), μ (10⁻³ Pa·s), and ϕ (0.6) were held constant; Rₚ and Rₘ were varied from 1 to 3 μm and 1 to 3 mm, respectively. The results, along with regimes of Pe<1 calculated from Eq. 2, are plotted in FIG. 3B.

$$Δ P = \frac{8 μ {Q}_{p} {δ}_{m}}{π {R}_{p}^{4}} = \frac{8 μ Q {δ}_{m}}{π ∅ {R}_{p}^{2} {R}_{m}^{2}} \qquad ( S \text{-} 8 )$$

### IX. DNA BINDING EFFICIENCY AS A FUNCTION OF PE

100 ng of salmon sperm DNA (Invitrogen, CA) in 200 μL of 10 mM MES buffer (pH ˜5) was flushed through a 4 mm chitosan membrane at different flow rates via the syringe/luer lock system shown in FIGS. 8A-E. The inlet and eluate DNA concentration of each flush was measured with PicoGreen dye (Invitrogen, CA); converting to mass (m_{DNA}), Eq. S-9 was then used to calculate the capture efficiency.

$$Capture \% = ( \frac{{m}_{D N A , out}}{{m}_{D N A , i n}} ) \cdot 100 \qquad ( S \text{-} 9 )$$

Pe was calculated via Eq. 2 and the results are plotted in FIG. 4. This agrees with theoretical predictions that Pe>1 results in reduced capture. Also, layering the nylon membrane with chitosan does not significantly hinder flow rate or require untenable pressure drops to achieve flow rates of ˜1 mL/min and efficient capture.

### X. COMPATIBILITY OF CHITOSAN MEMBRANE WITH IN SITU AMPLIFICATION

To test the compatibility of chitosan membranes with in situ PCR amplification, 1 μL of varying concentrations of λ DNA was wetted into a 4 mm diameter chitosan membrane. The membrane was then placed in a well plate and 10 μL PCR mix was added to the well. Replicates containing 10 μL PCR mix with the same amount of λ DNA and no membrane present were also included. The well plate was inserted into an Ilumina Eco™ real-time PCR System (EC-101-1001) and thermal cycled; correct λ-phage DNA product was verified with melt curve analysis. The PCR mix and thermal cycling conditions used were the same as described in the “Capture and In situ Amplification” Section. FIG. 6A shows that chitosan membranes are compatible with in situ PCR amplification down to ˜2 copies/reaction.

LAMP reagents were purchased from Eiken Chemical (Tokyo, Japan), product code LMP207. The LAMP mixture used for amplification of λ-phage DNA contained the following: 5 μL Reaction Mixture, 0.4 μL of Enzyme Mixture, 0.5 μL of 20× LAMP primer mixture (Table S-7), 1.25 μL of Calcein (Fd), and 3.85 μL of nuclease-free water.

### XI. CAPTURE AND IN SITU AMPLIFICATION

Provided in Table S-4 are volumes of 10 mM MES (2-(N-morpholino)ethanesulfonic acid) buffer and final concentrations of λ DNA used for FIG. 9B. In all of these experiments, 100 ng or less of salmon sperm DNA was added to the solution as background DNA.

TABLE S-4

| | Volume | | | |
|---|---|---|---|---|
| | of 10 mM | | | |
| Copies | MES | Concen- | | Total |
| of λ | buffer | tration | Positive | membranes |
| DNA | (mL) | (cop/mL) | membranes | tested |
| 9 | 10 | 0.9 | 6 | 6 |
| 10 | 10 | 1.0 | 3 | 3 |
| 9 | 5 | 1.8 | 6 | 6 |
| 6 | 3 | 2.0 | 2 | 3 |
| 10 | 5 | 2.0 | 2 | 3 |
| 6 | 1 | 6.0 | 3 | 3 |

Provided in table S-5 are volumes of 10 mM MES (2-(N-morpholino)ethanesulfonic acid) buffer, final concentrations of k DNA, and amounts of background DNA added for FIG. 9A. In these experiments, salmon sperm DNA was used as the “background DNA”.

TABLE S-5

| | Volume | | Back- | | |
|---|---|---|---|---|---|
| | of 10 mM | λ DNA | ground | | |
| Copies | MES | Concen- | DNA | | Total |
| of λ | buffer | tration | added | Positive | membranes |
| DNA | (mL) | (cop/mL) | (ng) | membranes | tested |
| 25 | 50 | 0.5 | 100 | 6 | 10 |
| 25 | 50 | 0.5 | 10 | 9 | 9 |

To detect λ DNA product after in situ amplification, two methods were used. i) After thermal cycling the membrane with PCR mix in a well plate, an appropriate amount of 6× gel loading dye and TE buffer was added to each well and pipette mixed. Then, 5 μL of this solution was removed from the well, placed in a 1.2% agarose gel, and run for 50 min at 80V. Samples with DNA product at the same length as the λ PCR amplicon (322 base pairs) were considered positive. An example of a gel image is shown in FIG. 10A. ii) After thermal cycling, the PCR reaction mixture was transferred to an empty well and an appropriate amount of 20× Evagreen dye (Biotium) and 10×TE buffer was added. A continuous melt curve was then obtained from 65-95° C.; samples with a peak around ˜85° C. (the melting temperature of the λ PCR amplicon) were considered positive (FIG. 10B).

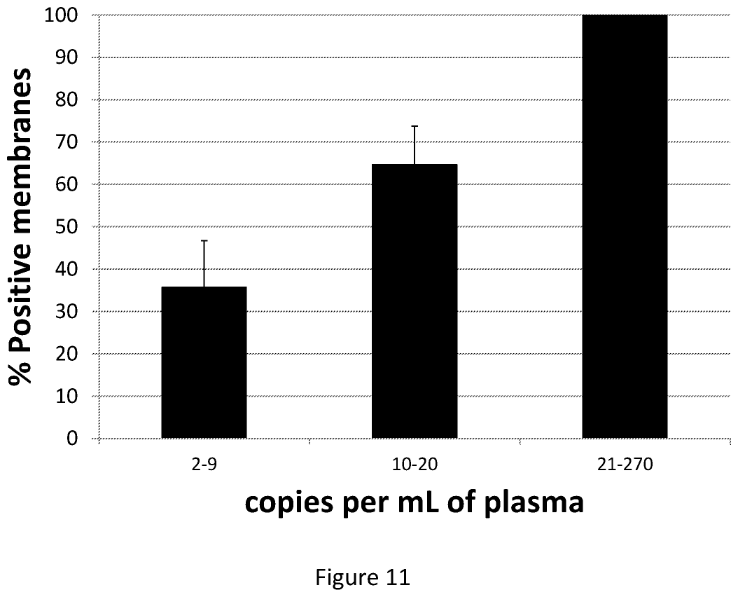

TABLE S-6
Volumes of plasma before lysis, lysed plasma, and
final concentrations of λ DNA used for FIG. 11.

| | | Volume | Concen- | | |
|---|---|---|---|---|---|
| Copies | Volume | of lysed | tration | | Total |
| of λ | of plasma | plasma | (cop/mL | Positive | membranes |
| DNA | (mL) | (mL) | plasma) | membranes | tested |
| 10 | 5 | 10 | 2 | 0 | 1 |
| 20 | 10 | 20 | 2 | 0 | 1 |
| 10 | 4 | 8 | 2.5 | 0 | 1 |
| 20 | 5 | 10 | 4 | 0 | 1 |
| 10 | 2 | 4 | 5 | 4 | 7 |
| 20 | 4 | 8 | 5 | 0 | 2 |
| 9 | 1 | 2 | 9 | 1 | 1 |
| 10 | 1 | 2 | 10 | 7 | 11 |
| 18 | 1 | 2 | 18 | 1 | 1 |
| 20 | 1 | 2 | 20 | 3 | 5 |
| 30 | 1 | 2 | 30 | 4 | 4 |
| 45 | 1 | 2 | 45 | 1 | 1 |
| 90 | 1 | 2 | 90 | 1 | 1 |
| 270 | 1 | 2 | 270 | 1 | 1 |

### XII. Λ-PHAGE DNA PCR AMPLIFICATION AND A-PHAGE DNA LAMP AMPLIFICATION

A mixture of primers from Table S-7 was made at 5 μM each in nuclease-free water and used for the PCR amplification reactions described in this manuscript.

TABLE S-7
Sequences for λ-phage DNA PCR primers.

| | | |
|---|---|---|
| forward | CGTTGCAGCAATATCTGGGC | SEQ. ID NO. 1 |
| reverse | TATTTTGCATCGAGCGCAGC | SEQ. ID NO. 2 |

A mixture of each primer from Table S-8 was made in nuclease-free water and used for the LAMP amplification reactions described in S-IV. The concentration of each primer in the 20× mixture is also listed.

TABLE S-8
Sequences for λ-phage DNA LAMP primers⁵¹ and their concentration in the
20X primer mix.

| | | | SEQ. |
|---|---|---|---|
| Name | Sequence | Conc. | ID NO. |
| FOP | GGCTTGGCTCTGCTAACACGTT | 4 μM | 3 |
| BOP | GGACGTTTGTAATGTCCGCTCC | 4 μM | 4 |
| FIP | CAGCCAGCCGCAGCACGTTCGCTCATAGGAGATATGGTAGAGCCGC | 32 μM | 5 |
| BIP | GAGAGAATTTGTACCACCTCCCACCGGGCACATAGCAGTCCTAGGGACAGT | 32 μM | 6 |
| LOOPF | CTGCATACGACGTGTCT | 8 μM | 7 |
| LOOPR | ACCATCTATGACTGTACGCC | 8 μM | 8 |

### XIV. CONCLUSION

An approach was evaluated for ultrasensitive detection of nucleic acids using chitosan as a charge-switch matrix that enables concentration factors up to 5000× and subsequent in situ amplification. A theoretical model guided the parameters chosen for flow rate, membrane radius, and pore radius. Based on model predictions, membranes with specific pore and membrane radii were functionalized to capture low copy numbers of nucleic acids from large volumes in short times. Using this approach, zeptomolar concentrations of nucleic acids were captured from up to 50 mL of solution at a flow rate of 1 mL/min with ΔP<1 atm. In applications with different requirements for flow rate, pressure drop, or membrane size, this theory can be applied to guide choices of membrane parameters that meet those requirements.

In addition, flowing through a matrix that is compatible with in situ amplification obviates the need for centrifugation or bead manipulation and simplifies the purification process by eliminating an elution step. Chitosan-coated nylon membranes are sturdy, flexible, and small enough to be incorporated into integrated devices for complete sample-to-answer diagnostics. In this study, the theory and the proof-of-principle experiments using solutions of purified nucleic acids in clean matrixes were focused on. However, more complex matrices are encountered in many applications. Ultrasensitive measurements of viral, bacterial, and cancer-associated nucleic acids provide important diagnostic information to clinicians, but require the extraction and detection of NAs from milliliters of plasma and in some cases cell lysis. The described methods can also be used for detection from a variety of sample matrices, such as blood, plasma, urine and water. The described methods can also be applied with isothermal amplification which in turn enables rapid and ultra-sensitive nucleic acid measurements for point-of-care and limited-resource settings.

### REFERENCES

All publications and patents cited in this specification are herein, including those listed below, are incorporated by reference as if each individual publication or patent were specifically and individually indicated to be incorporated by reference and are incorporated herein by reference to disclose and describe the devices, methods and/or materials in connection with which the publications are cited.
(1) World Health Organization. Guidelines for drinking-water quality: recommendations; 2004; Vol. 1.
(2) Fong, T.-T.; Lipp, E. K. Microbiol. Mol. Biol. Rev. 2005, 69, 357-371.
(3) Sarmati, L.; D'Ettorre, G.; Parisi, S. G.; Andreoni, M. Current HIV Research 2015, 13, 250.
(4) Raboud, J. M.; Montaner, J. S.; Conway, B.; Rae, S.; Reiss, P.; Vella, S.; Cooper, D.; Lange, J.; Harris, M.; Wainberg, M. A. AIDS 1998, 12, 1619-1624.
(5) Hodgson, S. H.; Douglas, A. D.; Edwards, N. J.; Kimani, D.; Elias, S. C.; Chang, M.; Daza, G.; Seilie, A. M.; Magiri, C.; Muia, A. Malar. J. 2015, 14, 33.
(6) Jiang, S.; Noble, R.; Chu, W. Appl. Environ. Microbiol. 2001, 67, 179-184.
(7) Haramoto, E.; Kitajima, M.; Katayama, H.; Ohgaki, S. water research 2010, 44, 1747-1752.
(8) Campana, D.; Pui, C.-H. BLOOD-NEW YORK—1995, 85, 1416-1416.
(9) Maggiolo, F.; Callegaro, A.; Cologni, G.; Bernardini, C.; Velenti, D.; Gregis, G.; Quinzan, G.; Soavi, L.; Iannotti, N.; Malfatto, E. JAIDS Journal of Acquired Immune Deficiency Syndromes 2012, 60, 473-482.
(10) Castillo, I.; Bartolome, J.; Quiroga, J.; Barril, G.; Carrño, V. Aliment. Pharmacol. Ther. 2009, 30, 477-486.
(11) Keys, J. R.; Leone, P. A.; Eron, J. J.; Alexander, K.; Brinson, M.; Swanstrom, R. J. Med. Virol. 2014, 86, 473-477.
(12) Abubakar, A.; Ozumba, P.; Buttner, P.; Winter, J.; Abimiku, A. Journal of Antivirals & Antiretrovirals 2015, 2015.
(13) Yager, P.; Domingo, G. J.; Gerdes, J. Annu. Rev. Biomed. Eng. 2008, 10, 107-144.
(14) Urdea, M.; Penny, L. A.; Olmsted, S. S.; Giovanni, M. Y.; Kaspar, P.; Shepherd, A.; Wilson, P.; Dahl, C. A.; Buchsbaum, S.; Moeller, G. Nature 2006, 444, 73-79.
(15) Boom, R.; Sol, C.; Salimans, M.; Jansen, C.; Wertheim-van Dillen, P.; Van der Noordaa, J. J. Clin. Microbiol. 1990, 28, 495-503.
(16) Wen, J.; Legendre, L. A.; Bienvenue, J. M.; Landers, J. P. Anal. Chem. 2008, 80, 6472-6479.
(17) Dineva, M. A.; Mahilum-Tapay, L.; Lee, H. Analyst 2007, 132, 1193-1199.
(18) Chomczynski, P.; Sacchi, N. Anal. Biochem. 1987, 162, 156-159.
(19) Miller, D.; Bryant, J.; Madsen, E.; Ghiorse, W. Appl. Environ. Microbiol. 1999, 65, 4715-4724.
(20) Byrnes, S. A.; Bishop, J. D.; Lafleur, L.; Buser, J.; Lutz, B.; Yager, P. Lab on a Chip 2015, 15, 2647-2659.
(21) Cao, W.; Easley, C. J.; Ferrance, J. P.; Landers, J. P. Anal. Chem. 2006, 78, 7222-7228.
(22) Pandit, K. R.; Nanayakkara, I. A.; Cao, W.; Raghavan, S. R.; White, I. M. Anal. Chem. 2015, 87, 11022-11029.
(23) Hagan, K. A.; Meier, W. L.; Ferrance, J. P.; Landers, J. P. Anal. Chem. 2009, 81, 5249-5256.
(24) Kendall, E. L.; Wienhold, E.; DeVoe, D. L. Biomicrofluidics 2014, 8, 044109.
(25) Connelly, J. T.; Rolland, J. P.; Whitesides, G. M. Anal. Chem. 2015, 87, 7595-7601.
(26) Rodriguez, N. M.; Linnes, J. C.; Fan, A.; Ellenson, C. K.; Pollock, N. R.; Klapperich, C. M. Anal. Chem. 2015, 87, 7872-7879.
(27) Liu, C.; Geva, E.; Mauk, M.; Qiu, X.; Abrams, W. R.; Malamud, D.; Curtis, K.; Owen, S. M.; Bau, H. H. Analyst 2011, 136, 2069-2076.
(28) Rodriguez, N. M.; Wong, W. S.; Liu, L.; Dewar, R.; Klapperich, C. M. Lab on a Chip 2016.
(29) Reedy, C. R.; Bienvenue, J. M.; Coletta, L.; Strachan, B. C.; Bhatri, N.; Greenspoon, S.; Landers, J. P. Forensic science international: genetics 2010, 4, 206-212.
(30) Jangam, S. R.; Yamada, D. H.; McFall, S. M.; Kelso, D. M. J. Clin. Microbiol. 2009, 47, 2363-2368.
(31) Breadmore, M. C.; Wolfe, K. A.; Arcibal, I. G.; Leung, W. K.; Dickson, D.; Giordano, B. C.; Power, M. E.; Ferrance, J. P.; Feldman, S. H.; Norris, P. M. Anal. Chem. 2003, 75, 1880-1886.
(32) Shim, S.; Shim, J.; Taylor, W. R.; Kosari, F.; Vasmatzis, G.; Ahlquist, D. A.; Bashir, R. Biomedical microdevices 2016, 18, 1-8.
(33) Shen, F.; Du, W.; Kreutz, J. E.; Fok, A.; Ismagilov, R. F. Lab Chip 2010, 10, 2666-2672.
(34) Lukacs, G. L.; Haggie, P.; Seksek, O.; Lechardeur, D.; Freedman, N.; Verkman, A. J. Biol. Chem. 2000, 275, 1625-1629.
(35) Wink, T.; de Beer, J.; Hennink, W. E.; Bult, A.; van Bennekom, W. P. Anal. Chem. 1999, 71, 801-805.
(36) Hansen, R.; Bruus, H.; Callisen, T. H.; Hassager, O. Langmuir 2012, 28, 7557-7563.
(37) Koyayashi, T.; Laidler, K. J. Biotechnol. Bioeng. 1974, 16, 99-118.
(38) Nguyen, T. H.; Elimelech, M. Biomacromolecules 2007, 8, 24-32.
(39) Minehan, D. S.; Marx, K. A.; Tripathy, S. K. Macromolecules 1994, 27, 777-783.
(40) Chung, Y.-C.; Jan, M.-S.; Lin, Y.-C.; Lin, J.-H.; Cheng, W.-C.; Fan, C.-Y. Lab Chip 2004, 4, 141-147.
(41) Thömmes, J.; Kula, M. R. Biotechnol. Prog. 1995, 11, 357-367.
(42) Unarska, M.; Davies, P.; Esnouf, M.; Bellhouse, B. J. Chromatogr. A 1990, 519, 53-67.
(43) Wang, W.; Wang, H. B.; Li, Z. X.; Guo, Z. Y. Journal of Biomedical Materials Research Part A 2006, 77, 28-34.
(44) Linnes, J.; Rodriguez, N.; Liu, L.; Klapperich, C. Biomedical Microdevices 2016, 18, 1-12.
(45) Kyung-Hee, C.; Kwang-hee, K. P. Bulletin of the Korean Chemical Society 1983, 4, 68-72.
(46) Nagamine, K.; Hase, T.; Notomi, T. Mol. Cell. Probes 2002, 16, 223-229.
(47) Thömmes, J.; Kula, M. R. Biotechnol. Prog. 1995, 11, 357-367.
(48) Lukacs, G. L.; Haggie, P.; Seksek, O.; Lechardeur, D.; Freedman, N.; Verkman, A. J. Biol. Chem. 2000, 275, 1625-1629.
(49) Wink, T.; de Beer, J.; Hennink, W. E.; Bult, A.; van Bennekom, W. P. Anal. Chem. 1999, 801-805.
(50) Kamm, R. C.; Smith, A. G. Clin. Chem. 1972, 18, 519-522.
(51) Nagamine, K.; Hase, T.; Notomi, T. Mol. Cell. Probes 2002, 16, 223-229.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of detecting a low concentration analyte in a sample, the method comprising:
a. flowing a sample comprising the low concentration analyte through a porous membrane coated with a capture matrix and thereby capturing analyte on the coated membrane; and
b. detecting the captured analyte,
wherein the analyte has a concentration within the sample of 500 entities/mL or less, and wherein the flowing is performed in 1 hour or less.

2. The method of claim 1, wherein the analyte has a concentration within the sample of 100 entities/mL or less.

3. The method of claim 1, wherein the analyte has a concentration within the sample of 10 units/mL or less.

4. The method of claim 1, wherein the flowing is performed in 30 min or less.

5. The method of claim 1, wherein the flowing is performed in 10 min or less.

6. The method of claim 1, wherein the sample is flowed through the coated membrane at a rate of 0.1 mL/minute or greater, 0.5 mL/minute or greater, or 1 mL/minute or greater.

7. (canceled)

8. (canceled)

9. The method of claim 1, wherein the sample has a volume of 0.1 mL or greater, 1 mL or greater, or 20 mL or greater.

10. (canceled)

11. (canceled)

12. The method of claim 1, wherein the analyte comprises nucleic acids.

13. The method of claim 1, wherein detecting the captured analyte comprises performing nucleic acid amplification.

14. The method of claim 13, wherein the coated membrane is in a container and the nucleic acid amplification is performed while the captured analyte is in the container.

15. The method of claim 1, wherein the coated membrane comprises a matrix comprising a polymeric material.

16. The method of claim 15, wherein the coated membrane comprises chitosan.

17. The method of claim 15, wherein the polymeric material comprises poly-L-lysine.

18. The method of claim 1, wherein flowing the sample through the coated membrane comprises concentrating the sample on the membrane by 1000× or more.

19.-21. (canceled)

22. A method of performing in-situ amplification on a sample, the method comprising:
a. flowing the sample comprising a first concentration of an analyte through a porous membrane coated with a capture matrix in a container and thereby capturing analyte with the coated membrane to provide a captured sample comprising a second concentration of analyte which is 1000× or more than the first concentration; and
b. amplifying the analyte within the container,
wherein the flowing and amplifying are performed in 1 hour or less.

23.-43. (canceled)

44. The method of claim 1, wherein the membrane is cylindrical and has a membrane radius of 2 mm or less.

45. The method of claim 44, wherein the membrane has a pore radius ranging from 0.5 to 20 μm.

46. The method of claim 45, wherein the membrane has a thickness ranging from 0.3 to 3500 μm.

47.-49. (canceled)

50. A low concentration analyte capture device, the device comprising:
a. a housing;
b. a porous membrane coated with a capture matrix, said coated membrane operatively coupled to the housing and configured to capture and thereby concentrate analyte from a sample flowed therethrough by 1000× or more in 30 min or less.

51. The device of claim 50, wherein the housing comprises a container and the coated membrane is positioned within the container.
